(19) United States
(12) Patent Application Publication
(10) Pub. No.: US 2023/0226228 A1
(43) Pub. Date: Jul. 20, 2023

(54) POSITRON IMAGING TOMOGRAPHY IMAGING AGENT COMPOSITION AND METHOD FOR DETECTION OF BACTERIAL INFECTION

(71) Applicant: The Research Foundation for the State University of New York, Albany, NY (US)

(72) Inventors: Peter J. Tonge, Stony Brook, NY (US); Yong Li, Stony Brook, NY (US); Fareidoon Daryaee, Stony Brook, NY (US); Peter Michael Smith-Jones, Stony Brook, NY (US); Labros Meimetis, Stony Brook, NY (US)

(73) Assignee: The Research Foundation for the State University of New York, Albany, NY (US)

(21) Appl. No.: 17/997,908

(22) PCT Filed: May 4, 2021
(86) PCT No.: PCT/US2021/030642

§ 371 Date: Nov. 3, 2022

Related U.S. Application Data
(60) Provisional application No. 63/019,685, filed on May 4, 2020.

(51) Int. Cl. A61K 51/04; C07C 201/12; A61P 31/04

(52) U.S. Cl. CPC A61K 51/0402; C07C 201/12; A61P 31/04

(57) ABSTRACT

This invention provides a composition comprising the compound having the structure:

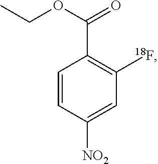

ethyl 2-[¹⁹F]F-4-nitrobenzoate, and at least one acceptable carrier.

This invention also provides a method of detecting the presence of or location of bacteria cells in a subject which comprises determining if an amount of the compound or determining where an amount of the compound having the structure:

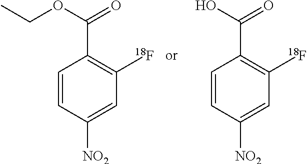

is present in the subject at a period of time after administration of the compound or salt thereof to the subject, thereby detecting the presence of or location of the bacteria cells based on the amount of the compound determined to be present in the subject or detecting the location of the bacteria cells based on the location of the compound determined to be present in the subject.

This application claims priority of U.S. Provisional Application No. 63/019,685, filed May 4, 2020, the contents of which are hereby incorporated by reference.

### GOVERNMENT SUPPORT

This invention was made with government support under HL127522, EB024549 and EB027050 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Treatment of bacterial infections in humans is hindered by the relatively unsophisticated diagnostic methods that are currently available, some of which are either slow or inaccurate and often depend on the availability of clinical samples that contain bacteria. Positron emission tomography (PET) is a promising non-invasive imaging technique for detecting and localizing infection in humans (Signore, A. & Glaudemans, A. W. 2011; Censullo, A. and Vijayan, T. 2017). Most of the current studies rely on fludeoxyglucose (FDG), however this tracer lacks specificity since it is taken up by any cell that is using glucose including human cells involved in the inflammatory response to infection (Jamar, F. et al. 2013; Meller, J. et al. 2007). Consequently, there is a need to develop sensitive and specific PET radiotracers for infection imaging.

2-Deoxy-2-[18F]fluoro-D-glucose ([¹⁸F]FDG) is one of the most widely studied and used PET tracers. It is an analog of glucose, and accumulates in tissues and organs that have high glycolytic activity such as tumor and brain cells. Therefore, FDG-PET is widely used in the detection and diagnosis of cancer and neurological disorders. FDG-PET has also been explored for the diagnosis of infection and indeed is used clinically for osteomyelitis, infective endocarditis, fever of unknown origin, tuberculosis and other infections (Jamar, F. et al. 2013; Meller, J. et al. 2007). However, due to the fact that FDG accumulates in all cells with high glycolytic activity including inflammatory cells and tumor cells, the ability of FDG-PET to correctly diagnose infection is limited, and results are often confounded by, for example, inflammation caused by other disorders as well as cancer.

Due to the lack of diagnostic sensitivity and specificity of FDG-PET, there have been several recent reports of novel infection tracers, including [¹²⁴I]FIAU (Bettegowda, C. et al. 2005), a small molecule substrate for bacterial thymidine kinase, [¹⁸F]fluorodeoxysorbitol (FDS) (Weinstein, E. A. et al. 2014; Li, Z. B. et al. 2008), and [¹⁸F]-labeled maltose based small molecules such as MH¹⁸F and 6-[¹⁸F]-fluoromaltose (Namavari, M. et al. 2015; Gowrishankar, G. et al. 2014). However each of these tracers face certain limitations for deployment as a S. aureus infection tracer, such as low signal-to-background ratio ([¹²⁴I]FIAU, 6-[¹⁸F]-fluoromaltose), low radiochemical yield (MH¹⁸F) or lack of uptake by S. aureus (FDS).

Recent efforts to develop bacteria specific and bacteria-class specific PET tracers are summarized in several detailed recent reviews (Ordonez, A. A. & Jain, S. K. 2018; Auletta, S. et al. 2019; Northrup, J. D. et al. 2019). One strategy to develop bacteria-specific radiotracers is to radiolabel compounds that are substrates for metabolic pathways that exist in bacteria but not in mammalian cells. In particular, the folate pathway is regarded as a promising imaging target to develop bacteria specific imaging tracers because folate must be biosynthesized by a wide range of bacteria. For example, ¹⁸F-fluoropropyl-trimethoprim (Sellmyer, M. A. et al. 2017), a ¹⁸F analogue of the bacterial dihydrofolate reductase (DHFR) inhibitor trimethoprim, and ¹¹C-p-aminobenzoic acid ([¹¹C]-PABA) (Mutch, C. A. et al 2018), a ¹¹C-labeled dihydropteroate synthase (DHPS) substrate, have been explored as potential radiotracers for imaging Gram positive and Gram negative bacterial infections.

2-[¹⁸F]F-p-aminobenzoic acid (2-[¹⁸F]F-PABA) is a bacteria-specific PET tracer that has been used to identify and localize S. aureus in a rodent soft tissue infection model (Zhang, Z. et al. 2018). In vitro enzymatic assays supported a mechanism for tracer accumulation in which bacterial dihydropteroate synthase (DHPS) catalyzes the incorporation of 2-[¹⁸F]F-PABA into folate. However, in vivo metabolite analysis demonstrated that 2-[¹⁸F]F-PABA underwent rapid N-acetylation, a transformation that would prevent the incorporation of the radiotracer into folic acid.

This invention describes two bacteria-specific PET tracers, Ethyl 2-[¹⁸F]F-4-Nitrobenzoate (2-[¹⁸F]F-ENB) and 2-[¹⁸F]F-4-Nitrobenzoate (2-[¹⁸F]F-NB), for use as a diagnostic tool for identifying and localizing S. aureus, which is a major cause of deep-seated infections.

### SUMMARY OF THE INVENTION

This invention provides a composition comprising the compound having the structure:

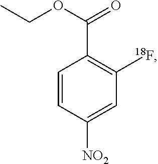

ethyl 2-[¹⁹F]F-4-nitrobenzoate, and at least one acceptable carrier.

This invention also provides a process for preparing the compound having the structure:

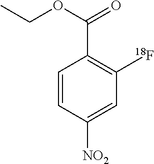

which comprises:
(a) reacting a compound having the structure:

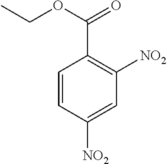 [SMILES: CCOC(=O)c1ccc([N+](=O)[O-])cc1[N+](=O)[O-]]

with a [¹⁸F] fluorinating agent to obtain the compound having the structure:

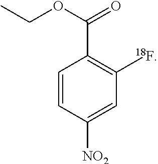

This invention also provides a process for preparing the compound having the structure:

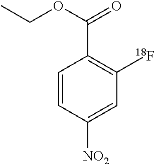

which comprises:

(a) reacting 2,4-dinitrobenzonitrile with a [¹⁸F] fluorinating agent to obtain the compound having the structure:

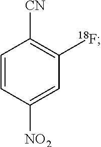

(b) reacting the compound obtained in step (a) with a base to obtain the compound having the structure:

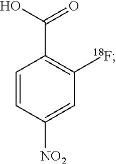

and

(c) esterifying the carboxylic acid group in the compound obtained in step (b) to obtain the compound having the structure:

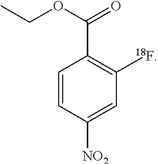

This invention also provides a method of detecting the presence of or location of bacteria cells in a subject which comprises determining if an amount of the compound or determining where an amount of the compound having the structure:

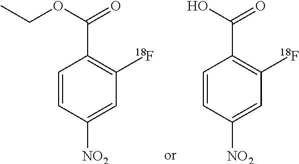

is present in the subject at a period of time after administration of the compound or salt thereof to the subject, thereby detecting the presence of or location of the bacteria cells based on the amount of the compound determined to be present in the subject or detecting the location of the bacteria cells based on the location of the compound determined to be present in the subject.

### BRIEF DESCRIPTION OF THE FIGURES

FIG. 1. HPLC trace of 2-[¹⁸F]F-ENB prepared manually and by automated synthesis. (a) Semi-preparative HPLC chromatography of manually-synthesized 2-[¹⁸F]F-ENB using a Phenomenex Luna 10 μm C18(2) column (250×10 mm) with 45% MeCN/55% H₂O as the eluent. The flow rate 4 mL/min. The blue trace is the UV absorption of the eluent at 287 nm and the red trace is the radioactive signal. (b) Analytical HPLC of a co-injection of the 2-F-ENB cold standard with purified 2-[¹⁸F]F-ENB prepared by manual synthesis. The blue trace is the UV absorption of the eluent at 287 nm, and the red trace is the radioactive signal. (c) Analytical HPLC chromatography of purified 2-[¹⁸F]F-ENB prepared by automated synthesis using a Phenomenex Luna 10 μm C18(2) column (250×10 mm) with 45% MeCN/55% H₂O as the eluent. The flow rate 0.8 mL/min. The blue trace is the UV absorption of the eluent at 254 nm, and the red trace is the radioactive signal. (d) Analytical HPLC of a co-injection of the 2-F-ENB cold standard with purified 2-[¹⁸F]F-ENB prepared by automated synthesis. The blue trace is the UV absorption of the eluent at 254 nm, and the red trace is the radioactive signal.

FIG. 2. Histology of S. aureus Newman infection and sterile inflammation in the rat model. (a) H&E staining of S. aureus-infected triceps. (b) H&E staining of triceps with sterile inflammation (heat-killed S. aureus). (c) Gram stain of infected triceps showing Gram-positive cocci in clusters. (d) Gram stain of inflamed triceps showing that no Gram-positive cocci are found.

FIG. 3. PET/CT imaging of a rat model of S. aureus Newman-induced triceps infection. (A) [¹⁸F]FDG PET/CT of a rat in which 8.2±0.2 log₁₀ CFU of S. aureus Newman has been injected into the right triceps (red arrow) and with sterile inflammation in the left triceps (yellow arrow). The image was acquired 60-80 min after tracer injection. (B) Comparison of [18F]FDG accumulation 60-80 min postinjection at sites of infection and sterile inflammation, represented as % injected dose per cc (% ID/cc). (C) 2-[¹⁸F]F-ENB accumulation was observed in the infected (red arrow) but not inflamed triceps (yellow arrow) 180-200 min after tracer injection. (D) Comparison of 2-[¹⁸F]F-ENB accumulation in the infected triceps and inflamed triceps 180-200 min after tracer injection: ***P<0.001 from a two-tailed Mann-Whitney U Test (n=7). (E) Ex vivo PET imaging of the excised infected triceps. The area of infection is highlighted in yellow. (F) Post-mortem ex vivo analysis of 2-[¹⁸F]F-ENB biodistribution at sites of infection and sterile inflammation 3 h after tracer injection, represented as % injected dose per gram (% ID/g): ***P<0.001 from a two-tailed Mann-Whitney U Test (n=8).

FIG. 4. Post-mortem analysis of 2-[¹⁸F]F-ENB distribution. After the animals were injected IV with 2-[¹⁸F]F-ENB and scanned for 180 min, they were euthanized, and the tissues weighed and harvested for automated gamma counting. Data are medians with interquartile and ranges are shown (n=6).

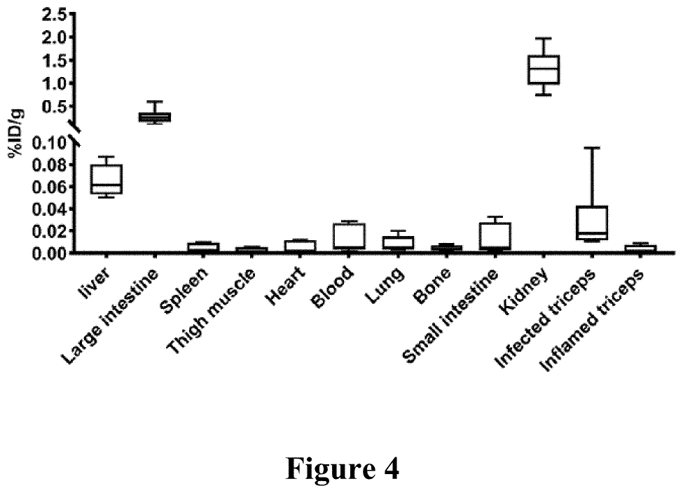

FIG. 5. PET/CT imaging of rats infected in the right triceps with the bioluminescent Xen29 strain of S. aureus. (A) Experimental design. (B) 2-[¹⁸F]F-ENB-derived radioactivity accumulates in the infected triceps (red arrow) but not the inflamed triceps (yellow arrow) at 3 h post tracer administration. (C) Ex vivo PET/CT imaging and bioluminescent imaging of the excised triceps demonstrated that the radioactivity in the infected triceps colocalized with the bioluminescence signal generated by S. aureus Xen29.

FIG. 6. In vitro metabolic stability of 2-[¹⁸F]F-ENB in rat and human plasma. (a-d) HPLC traces of metabolites of 2-[¹⁸F]F-ENB incubated with rat plasma at different time points (2 min, 10 min, 30 min, 85 min) and spiked with cold standards. (e) The metabolic half-life of 2-[¹⁸F]F-ENB in rat plasma was calculated by fitting the percentage of either intact tracer or metabolites generated in human plasma as the function of time to a single exponential equation. (f) The metabolic half-life of 2-[¹⁸F]F-ENB in human plasma was calculated by fitting the percentage of either intact tracer or metabolites generated in human plasma as the function of time to a single exponential equation. 2-[¹⁸F]F-ENB has longer half-life in human plasma (t_{1/2}=35 min) than in rat plasma (t_{1/2}=2 min).

FIG. 7. Analysis of metabolites in the plasma and urine of healthy rats after intravenous injection (IV) of 2-[¹⁸F]F-ENB. After IV administration of 2-[¹⁸F]F-ENB, the animals were sacrificed at each time point (n=3 rats) and the blood and urine samples were collected for radio-HPLC analysis. (A) A representative HPLC chromatogram is shown to illustrate the separation of four major nonradioactive cold standards: 2-F-PABA (a), 2-F-NB (b), 2-F-PABA ester (c), and 2-F-ENB (d). The red trace is the HPLC chromatogram of a blood sample that reveals the presence of 2-[¹⁸F]F-NB and 2-[¹⁸F]F-ENB. The percent of each metabolite in blood (B) and urine (C) is shown.

FIG. 8. PET/CT imaging of 2-[¹⁸F]F-NB in a rat model of S. aureus Xen29 infection. (A) Radiosynthesis of 2-[¹⁸F]F-NB. (i) [¹⁸F]potassium fluoride, Kryptofix₂₂₂, potassium carbonate, dimethyl sulfoxide, r.t., 10 min; (ii) 2 M potassium hydroxide in H₂O, 105° C., 10 min; Overall decay-corrected radiochemical yield: 26% (n=1). (B) Accumulation of 2-[¹⁸F]F-NB-derived radioactivity was observed in the infected (red arrow) but not inflamed triceps (yellow arrow). (C) Ex vivo bioluminescence and (D) PET/CT imaging of excised triceps showed that the radioactivity in the infected triceps colocalized with the bioluminescent signal emitted by the S. aureus Xen29 strain.

FIG. 9. Analytical HPLC of 2-[¹⁸F]F-NB prepared by automated synthesis. (a) Analytical HPLC chromatography of purified 2-[¹⁸F]F-NB prepared by automated synthesis using a Phenomenex Luna 10 μm C18(2) column (250×10 mm) with 25% MeCN/75% H₂O with 0.1% TFA as the eluent. The flow rate 0.8 mL/min. The blue trace is the UV absorption of the eluent at 254 nm, and the red trace is the radioactive signal. (b) Analytical HPLC of a co-injection of the 2-F-NB cold standard with purified 2-[¹⁸F]F-NB prepared by automated synthesis. The blue trace is the UV absorption of the eluent at 254 nm, and the red trace is the radioactive signal.

FIG. 10. Time activity curve (TAC) in infected triceps, inflamed triceps and blood and biodistribution of 2-[¹⁸F]F-NB in major organs. (a) The right triceps of the rats were infected with S. aureus Xen 29 and the left triceps were injected with a 10-fold higher burden of heat-killed S. aureus to induce sterile inflammation. The rats were scanned by PET/CT for 120 min (rats with sterile inflammation in the left triceps) immediately starting from tracer administration. The PET scan was separated into ten frames (1-min) and twenty-two frames (5-min). TACs of infected triceps (blue), inflamed triceps (grey) and blood (left heart ventricle, red) are shown. Data represented as mean with standard error of the mean (n=4). (b) Time-dependent accumulation of 2-[¹⁸F]F-NB in infected and inflamed triceps compared to the blood (left heart ventricle). Data are presented as the mean and standard error of the mean (n=4). (c) Comparison of 2-[¹⁸F]F-NB accumulation in the infected triceps and inflamed triceps 180-200 min after tracer injection (n=3). (d) Post-mortem ex vivo analysis of 2-[¹⁸F]F-NB biodistribution, represented as % injected dose per gram (% ID/g). After the animals were injected IV with 2-[¹⁸F]F-NB and scanned for 180 min, they were sacrificed and the tissues and major organs weighed and harvested for automated gamma counting. Data represented as mean with standard error of the mean (n=3).

FIG. 11. Kinetics of 2-F-ENB, 2-F-NB and 2-F-NAP reduction by NfsB. The kinetics for the reduction of ethyl 2-fluoro-p-nitrobenzoate (F-ENB) (a), 2-fluoro-p-nitrobenzoate (F-NB) (b) and 2-F-4-nitroacetophenone (F-NAP) (c). F-ENB, F-NB and F-NAP by NfsB was performed in 50 mM Tris-HCl buffer (pH 7.4) containing 5 mM EDTA, and 40 μM NADH in a total reaction volume of 500 μL. The reaction was initiated by adding enzyme (22 nM) and the consumption of NADH was monitored at 340 nm. The kcat and Km values were determined by varying the concentration of F-ENB (10-100 μM), F-NB (40-1000 μM) and F-NAP (10-100 μM). Initial velocities as a function of substrate concentration were fitted to the Michaelis-Menten equation using GraphPad Prism 4. Each experiment was performed in triplicate and all the data points were included when the data were fit by the Michaelis-Menten equation.

FIG. 12. In vitro 2-[¹⁸F]F-NB uptake by S. aureus Newman and Xen29. 1 μCi of 2-[¹⁸F]F-NB was incubated in a 1 mL culture of S. aureus Newman (green bars) or S. aureus Xen29 (blue bars) for 5, 30, 60, 90 and 120 min. At both 60 and 120 min, there was significantly higher uptake in live S. aureus Newman and S. aureus Xen 29 compared to heat killed (HK) S. aureus strains.

FIG. 13. Impact of PABA analogues on the antibacterial activity of sulfamethoxazole (SMX). (A) S. aureus Xen29 were incubated with SMX (0.048-100 μg/mL) either alone or in the presence of PABA (0.8 μg/mL and 4 μg/mL) demonstrating that PABA can rescue the inhibition of growth caused by SMX in a concentration-dependent manner. (B-D) F-PABA and 2-F-NB but not 2-F-NAP can rescue the inhibition of growth caused by SMX.

FIG. 14. A comparison of PET imaging of the triceps infection/inflammation model with 2-[¹⁸F]F-ENB (A) and 2-[¹⁸F]F-NB (B). Both radiotracers were shown to localize the S. aureus Xen29 infection (red arrow) with no accumulation at the site of inflammation (yellow arrow), 3 h post injection of radiotracers.

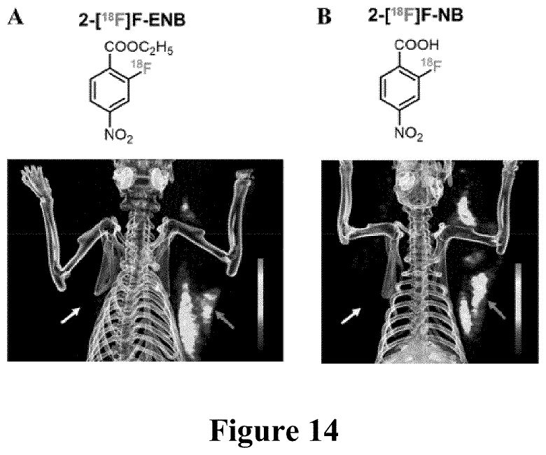

FIG. 15. The in vivo PET signal from the radiotracer 2-[¹⁸F]F-ENB are colocalized with bioluminescence signal (BLI) from the S. aureus in Rat 1. (A) In vivo bioluminescence. (B) In vivo PET image at 50-60 minutes. (C) In vivo PET image at 110-120 minutes.

FIG. 16. The in vivo PET signal from the radiotracer 2-[¹⁸F]F-ENB are colocalized with bioluminescence signal (BLI) from the S. aureus in Rat 2. (A) In vivo bioluminescence. (B) In vivo PET image at 50-60 minutes. (C) In vivo PET image at 110-120 minutes.

FIG. 17. The ex vivo PET signal from isolated heart is also colocalized with ex vivo bioluminescence signal in Rat 1. (A). (B) Ex vivo bioluminescence. (C) Ex vivo PET image.

FIG. 18. The ex vivo PET signal from isolated heart is also colocalized with ex vivo bioluminescence signal in Rat 2. (A) Ex vivo bioluminescence. (B) Ex vivo PET image.

FIG. 19. Ex vivo BioD analysis at 3-h post injection of radiotracer.

FIG. 20. The in vivo PET signal from the radiotracer 2-[¹⁸F]fluoro-4-nitro benzoate: 2-[¹⁸F]F-NB are colocalized with bioluminescence signal (BLI) from the S. aureus. (A) In vivo bioluminescence. (B) In vivo PET image at 1.5 h post-injection. (C) In vivo PET image at 2.5 h post-injection.

FIG. 21. Quantitative PET imaging and biodistribution analysis following intravenous injection of 2-[¹⁸F]F-NB.

FIG. 22. Ex vivo joint BLI and PET colocalization following intravenous injection of 2-[¹⁸F]F-NB (A) Ex vivo bioluminescence. (B) Ex vivo PET image.

### DETAILED DESCRIPTION OF THE INVENTION

The present invention provides a composition comprising the compound having the structure:

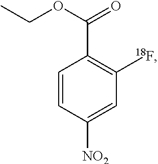

ethyl 2-[¹⁹F]F-4-nitrobenzoate, and at least one acceptable carrier.

In some embodiments, wherein the ratio of the ethyl 2-[¹⁸F]F-4-nitrobenzoate to the ethyl 2-[¹⁹F]F-4-nitrobenzoate is in a range from 1:90 to 1:550.

In some embodiments, wherein the ratio of the ethyl 2-[¹⁸F]F-4-nitrobenzoate to the ethyl 2-[¹⁹F]F-4-nitrobenzoate is in a range from 1:99 to 1:500.

In some embodiments, wherein the ratio of the ethyl 2-[¹⁸F]F-4-nitrobenzoate to the ethyl 2-[¹⁹F]F-4-nitrobenzoate is in a range from 1:324 to 1:500.

In some embodiments, wherein the ratio of the ethyl 2-[¹⁸F]F-4-nitrobenzoate to the ethyl 2-[¹⁹F]F-4-nitrobenzoate is in a range from 1:99 to 1:324.

In some embodiments, further comprising 4-amino-2-fluorobenzoic acid.

In some embodiments, wherein the ratio of the ethyl 2-[¹⁸F]F-4-nitrobenzoate to 4-amino-2-fluorobenzoic acid is about 1:99.

In some embodiments, wherein the ratio of the ethyl 2-[¹⁸F]F-4-nitrobenzoate to 4-amino-2-fluorobenzoic acid is about 1:100.

In some embodiments, wherein the ratio of the ethyl 2-[¹⁸F]F-4-nitrobenzoate to 4-amino-2-fluorobenzoic acid is about 1:134.

In some embodiments, wherein the ratio of the ethyl 2-[¹⁸F]F-4-nitrobenzoate to 4-amino-2-fluorobenzoic acid is about 1:500.

In some embodiments, wherein the radiochemical purity of ethyl 2-[¹⁸F]F-4-nitrobenzoate is at least 95%.

In some embodiments, wherein the radiochemical purity of ethyl 2-[¹⁸F]F-4-nitrobenzoate is at least 97.5%

In some embodiments, wherein the radiochemical purity of ethyl 2-[¹⁸F]F-4-nitrobenzoate is at least 99%.

In some embodiments, a process for preparing the composition of the present invention comprising admixing at least one carrier with an amount of a compound having the structure:

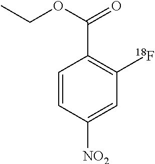

and ethyl 2-[¹⁹F]F-4-nitrobenzoate.

This invention also provides a process for preparing the compound having the structure:

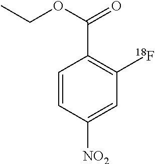

which comprises:

(a) reacting a compound having the structure:

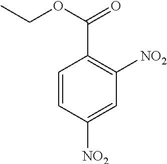 [SMILES: CCOC(=O)c1ccc([N+](=O)[O-])cc1[N+](=O)[O-]]

with a [¹⁸F] fluorinating agent to obtain the compound having the structure:

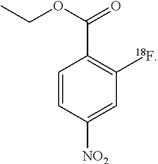

This invention also provides a process for preparing the compound having the structure:

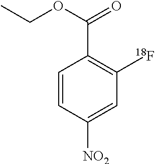

which comprises:

(a) reacting 2,4-dinitrobenzonitrile with a [¹⁸F] fluorinating agent to obtain the compound having the structure:

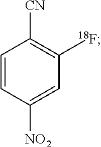

(b) reacting the compound obtained in step (a) with a base to obtain the compound having the structure:

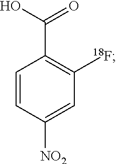

and

(c) esterifying the carboxylic acid group in the compound obtained in step (b) to obtain the compound having the structure:

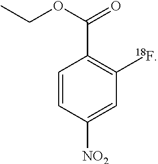

In some embodiments, wherein the [¹⁸F] fluorinating agent is potassium [¹⁸F] fluoride or tetra-n-butylammonium [¹⁸F] fluoride.

In some embodiments, wherein step (a) further comprises a chelating agent.

In some embodiments, wherein the chelating agent is a crown ether.

In some embodiments, wherein the chelating agent is 4,7,13,16,21,24-Hexaoxa-1,10-diazabicyclo[8.8.8]hexacosane.

In some embodiments, wherein step (a) further comprises a base.

In some embodiments, wherein the base is potassium carbonate.

In some embodiments, wherein step (a) is performed at 80-110° C.

In some embodiments, wherein in step (b) the hydrolysis is facilitated by aqueous solution of a base.

In some embodiments, wherein the base is potassium hydroxide.

In some embodiments, wherein step (b) is performed at a temperature of 80-110° C.

In some embodiments, wherein step (b) is performed at a temperature of about 105° C.

In some embodiments, wherein in step (c) the esterification is facilitated by a base.

In some embodiments, wherein the base is potassium hydroxide.

In some embodiments, wherein the base is sodium carbonate or potassium carbonate.

In some embodiments, wherein step (c) is performed at a temperature of 100-120° C.

In some embodiments, wherein step (c) is performed at a temperature of about 110° C.

In some embodiments, wherein in step (c) the esterification agent comprises a toluenesulfonyl group.

In some embodiments, wherein in step (c) the toluenesulfonyl group is ethyl tosylate.

In some embodiments, wherein in step (c) the esterification agent comprises a methylsulfonyl group.

In some embodiments, wherein in step (c) the methylsulfonyl group is ethyl mesylate.

In some embodiments, a composition comprising the compound having the structure:

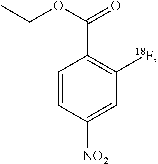

ethyl 2-[¹⁹F]F-4-nitrobenzoate, and at least one acceptable carrier,
wherein the compound is prepared by the process of the present invention.

This invention also provides a method of detecting the presence of or location of bacteria cells in a subject which comprises determining if an amount of the compound or determining where an amount of the compound having the structure:

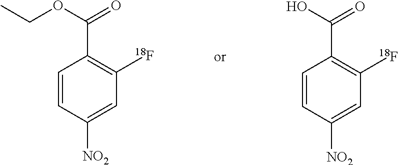

is present in the subject at a period of time after administration of the compound or salt thereof to the subject, thereby detecting the presence of or location of the bacteria cells based on the amount of the compound determined to be present in the subject or detecting the location of the bacteria cells based on the location of the compound determined to be present in the subject.

In some embodiments, further comprising quantifying the amount of the compound in the subject and comparing the quantity to a predetermined control.

In some embodiments, further comprising determining the level of infection in the subject based on the amount of the compound in the subject.

In some embodiments, wherein the determining is performed by a Positron Emission Tomography (PET) device.

In some embodiments, wherein the bacteria cells express dihydropteroate synthase (DHPS).

In some embodiments, wherein the subject is afflicted with a Gram-negative bacterial infection other than Enterococcus faecalis.

In some embodiments, wherein the subject is afflicted with a Gram-positive bacterial infection.

In some embodiments, wherein the subject is afflicted with a Mycobacterium tuberculosis bacterial infection.

In some embodiments, wherein the subject is afflicted with a Methicillin-sensitive Staphylococcus aureus bacterial infection.

In some embodiments, wherein the subject is afflicted with a Methicillin-resistant Staphylococcus aureus bacterial infection.

In some embodiments, further comprising subjecting the subject to antibiotic treatment when the presence of or location of bacteria cells is detected.

In some embodiments, wherein the antibiotic is a penicillin, a cephalosporin, a macrolide, a fluoroquinolone, a tetracycline, a carbapenem or an aminoglycoside antibiotic.

In some embodiments, wherein the subject is afflicted with osteomyelitis.

In some embodiments, wherein the subject is afflicted with endocarditis.

In some embodiments, wherein the subject is afflicted with a prosthetic joint infection.

In some embodiments, wherein the subject is afflicted with diabetes.

In some embodiments, wherein the subject is a mammal.

Ethyl 2-[¹⁸F]F-4-nitrobenzoate (2-[¹⁸F]F-ENB) has the following structure:

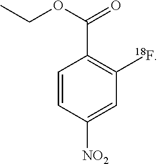

The present invention also provides a composition comprising 2-[¹⁸F]F-ENB.

The present invention also provides a composition comprising 2-[¹⁸F]F-ENB and a pharmaceutically acceptable carrier.

2-[¹⁸F]F-4-nitrobenzoate (2-[¹⁸F]F-NB) has the following structure:

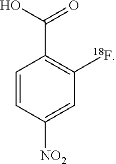

The present invention also provides a composition comprising 2-[¹⁸F]F-NB.

The present invention also provides a composition comprising 2-[¹⁸F]F-NB and a pharmaceutically acceptable carrier.

In some embodiments, a method for the detection of bacteria cells in a subject comprising:
(i) administering to the subject an effective amount of the composition of the present invention;
(ii) allowing a sufficient period of time for the bacteria cells to take up the 2-[¹⁸F]F-ENB or 2-[¹⁸F]F-NB in the composition; and
(iii) determining whether the bacteria cells are present in the host by detecting the 2-[¹⁸F]F-ENB or 2-[¹⁸F]F-NB the subject.

In some embodiments, a method of imaging bacteria cells of in a subject afflicted with an infection of the bacteria which comprises:
(i) administering to the subject an effective amount of the composition of the present invention;
(ii) imaging at least a portion of the subject;
(iii) detecting in the subject the location of the 2-[¹⁸F]F-ENB or 2-[¹⁸F]F-NB, thereby determining the location of the bacteria cells present in the subject based on the location of the 2-[¹⁸F]F-ENB or 2-[¹⁸F]F-NB in the subject;
(v) obtaining an image of the location of the bacteria cells; and optionally the following step:
(vi) repeating steps (i)-(v) one or more times.

In some embodiments, a method of determining the location of bacteria cells in a subject afflicted with an infection of the bacteria which comprises:
(i) administering to the subject an effective amount of the composition of the present invention;
(ii) allowing a sufficient period of time for bacteria cells in the subject to take up the 2-[¹⁸F]F-ENB or 2-[¹⁸F]F-NB;
(iii) imaging at least a portion of the subject; and
(iv) detecting in the subject the location of the 2-[¹⁸F]F-ENB or 2-[¹⁸F]F-NB, thereby determining the location of the bacteria cells present in the subject based on the location of the 2-[¹⁸F]F-ENB or 2-[¹⁸F]F-NB in the subject.

In some embodiments of any of the disclosed methods, the bacteria lacks a folate salvage pathway.

In some embodiments of any of the disclosed methods, the bacteria is methicillin-sensitive S. aureus (MSSA), methicillin-resistant S. aureus (MRSA), Gram-negative bacteria E. coli or Gram-negative bacteria Klebsiela pneumoniae or Mycobacterium tuberculosis.

In some embodiments of any of the disclosed methods, the 2-[¹⁸F]F-ENB or 2-[¹⁸F]F-NB accumulates in the cells of the bacteria by incorporation into the folate biosynthesis pathway.

In some embodiments of any of the disclosed methods, the 2-[¹⁸F]F-ENB or 2-[¹⁸F]F-NB is a substrate for DHPS.

In some embodiments of any of the disclosed methods, DHPS catalyses the condensation of 6-hydroxymethyl-7,8-dihydropteridine pyrophosphate to the 2-[¹⁸F]F-ENB or 2-[¹⁸F]F-NB to form 4-(((2-amino-4-oxo-3,4-dihydropteridin-6-yl)methyl)amino)-2-[¹⁸F]fluorobenzoic acid.

In some embodiments, the infectious bacteria is Gram-negative bacteria. In some embodiments, the infectious bacteria is Gram-positive bacteria. In some embodiments, the infectious bacteria is Gram-negative bacteria or Gram-positive bacteria.

In some embodiments, the infectious bacteria is Mycobacterium tuberculosis.

In some embodiments, the Gram-negative bacteria infection is drug-resistant or multi-drug resistant.

In some embodiments, the Gram-positive bacteria infection is drug-resistant or multi-drug resistant.

In some embodiments, the Mycobacterium tuberculosis infection is drug-resistant or multi-drug resistant.

In some embodiments, the Gram-negative bacteria cells are drug-resistant or multi-drug resistant.

In some embodiments, the Gram-positive bacteria cells are drug-resistant or multi-drug resistant.

In some embodiments, the method wherein the Mycobacterium tuberculosis cells are drug-resistant or multi-drug resistant.

In some embodiments, the Gram-negative bacteria is Escherichia coli, Klebsiella pneunomiae, Burkholderia cepacia, Pseudomonas aeruginosa or Acinetobacter baumanii.

In some embodiments, the Gram-negative bacteria is other than Enterococcus faecalis.

In some embodiments, the Gram-positive bacteria is Enterococcus faecalis, Enterococcus faecium, Staphylococcus aureus, Staphylococcus epidermidis, Staphylococcus haemolyticus, Staphylococcus lugdunensis, Staphylococcus saprophyticus, Staphylococcus hominis, Staphylococcus capitis, Streptococcus intermedius Streptococcus anginosus, Streptococcus constellatus, Streptococcus pneumoniae, Streptobacillus moniliformis, Streptococcus pyogenes, Streptococcus agalactiae, Actinomyces israelii, Arcanobacterium haemolyticum, Bacillus anthracis, Bacillus cereus, Bacillus subtilis, Clostridium difficile, Clostridium perfringens, Clostridium tetani, Corynebacterium diphtheria, Corynebacterium jeikeium, Corynebacterium urealyticum, Erysipelothrix rhusiopathiae, Listeria monocytogenes, Nocardia asteroides, Nocardia brasiliensis, Propionibacterium acnes or Rhodococcus equi.

In some embodiments, wherein the antibiotic includes, but is not limited to, fluoroquinolines, tetracyclines, macrolides, glycopeptides, sulfonamides, aminoglycosides, cephalosporins and/or penicillins.

In some embodiments, wherein the antibiotic is selected from the group consisting of ampicillin, piperacillin, penicillin G, ticarcillin, imipenem, meropenem, azithromycin, erythromycin, aztreonam, cefepime, cefotaxime, ceftriaxone, ceftazidime, ciprofloxacin, levofloxacin, clindamycin, doxycycline, gentamycin, amikacin, tobramycin, tetracycline, tigecycline, rifampicin, vancomycin and polymyxin.

In some embodiments, wherein the antibiotic is selected from the group consisting of gentamicin, amikacin, tobramycin, ciprofloxacin, levofloxacin, ceftazidime, cefepime, cefoperazone, cefpirome, ceftobiprole, carbenicillin, ticarcillin, mezlocillin, azlocillin, piperacillin, meropenem, imipenem, doripenem, polymyxin B, colistin and aztreonam.

In some embodiments, the subject is afflicted with osteomyelitis.

In some embodiments, the subject is afflicted with endocarditis.

In some embodiments, the subject is afflicted with a prosthetic joint infection.

In some embodiments, the subject is afflicted with diabetes.

As used herein, a “symptom” associated with a disease or disorder includes any clinical or laboratory manifestation associated with the disease or disorder and is not limited to what the subject can feel or observe.

As used herein, “treating”, e.g. of an infection, encompasses inducing prevention, inhibition, regression, or stasis of the disease or a symptom or condition associated with the infection.

As used herein, “radiochemical purity” or “RCP” is defined as the ratio of the activity of a radionuclide in a stated chemical species in a material over the total activity of all species containing that radionuclide in this material.

In some embodiments, the [¹⁸F] fluorinating agent is potassium [¹⁸F] fluoride.

In some embodiments, the [¹⁸F] fluorinating agent is tetra-n-butylammonium [¹⁸F] fluoride.

In some embodiments, the chelating agent is 4,7,13,16,21,24-Hexaoxa-1,10-diazabicyclo[8.8.8]hexacosane.

As used herein, Kryptofix₂₂₂ or Kryptofix 222 or Kryptofix [2.2.2] refers to 4,7,13,16,21,24-Hexaoxa-1,10-diazabicyclo[8.8.8]hexacosane.

In some embodiments, the base is potassium hydroxide. In some embodiments, the base is potassium carbonate. In some embodiments, the base is potassium bicarbonate. In some embodiments, the base is sodium hydroxide. In some embodiments, the base is sodium carbonate. In some embodiments, the base is sodium bicarbonate.

In some embodiments, the esterification agent is ethyl tosylate. In some embodiments, the esterification agent is ethyl mesylate.

As used herein, “ethyl tosylate” refers to ethyl p-toluenesulfonate.

As used herein “ethyl mesylate” refers to ethyl methanesulfonate.

As used herein, “about” mean±5%.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described—in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### EXAMPLE 1

### Radiosynthesis of 2-[¹⁸F]F-ENB

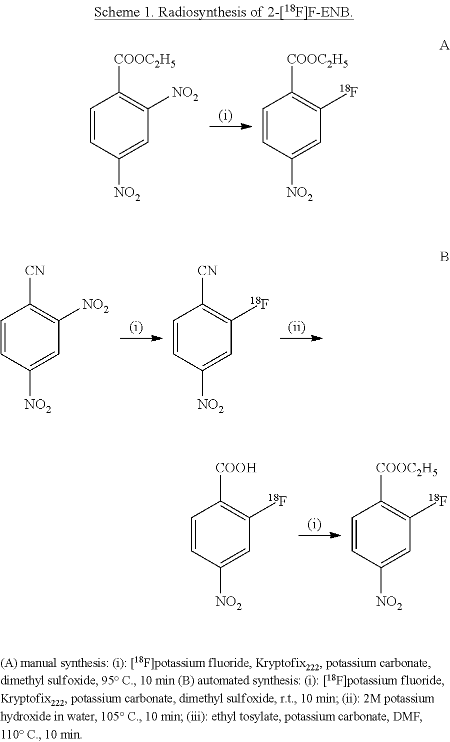

Manual Radiosynthesis of Ethyl 2-[¹⁸F]F-4-Nitrobenzoate (2-[¹⁸F]F-ENB). The radiosynthesis of 2-[¹⁸F]F-ENB was performed manually. Aqueous [¹⁸F]fluoride in ddH₂O was trapped on a QMA cartridge and then eluted with a solution containing Kryptofix K_{2.2.2} (7 mg) and potassium bicarbonate (2 mg) in 2 mL of acetonitrile (MeCN). The solvent was azeotropically evaporated under Argon, and 3 aliquots of MeCN (1 mL each) were successively added to the reaction vessel and then evaporated to afford a dry residue. The solid residue was resolubilized with 0.5 mL DMSO containing precursor (5 mg). The reaction vessel was securely capped and the reaction mixture was stirred and heated at 95° C. for 10 min and subsequently diluted with H₂O and loaded onto a preconditioned Waters C18 Light SepPak cartridge. The crude reaction product was eluted from the SepPak cartridge with MeCN (0.5 mL) into a receiving vial preloaded with H₂O (0.5 mL). The resulting mixture was then loaded onto a semipreparative HPLC column (Phenomenex Luna C18(2), 10 μm, 250×10 mm) and eluted with 45% MeCN/55% H₂O at a flow rate of 4 mL/min. The product fraction (eluting at 19-21 min) was collected and diluted with H₂O (30 mL). The diluted product solution was then passed through a second C18 Light SepPak cartridge. The final product was eluted from the SepPak with ethanol (0.3 mL) into a product vial precharged with USP saline (2.2 mL). Finally, the formulated product solution was passed through a membrane filter (Millipore Millex GV, 0.22 μm) into a vented sterile vial. A portion of the product solution was then taken to spike in the reference compound solution and analyzed with analytic HPLC (Phenomenex Luna C18(2), 10 μm, 250×4 mm, mobile phase: 45% MeCN/55% H₂O, flow rate: 0.8 mL/min) for quality control analysis.

Automated Radiosynthesis of Ethyl 2-[¹⁸F]F-4-Nitrobenzoate (2-[¹⁸F]F-ENB). [¹⁸F]F-ENB was synthesized using a GE Tracerlab FXN pro radiosynthesis box. [¹⁸F]Fluoride was produced at an offsite cyclotron and arrived at our facility around 4 h EOB. The [¹⁸F] fluoride was trapped on a WatersSepPak light Accell plus QMA cartridge and then eluted with 1 mL of 96% MeCN containing potassium carbonate (4 mg/mL)/Kryptofix K_{2.2.2} (14.4 mg/mL) into the reaction vessel. This solution was evaporated to dryness under a stream of nitrogen at 70° C. for 7 min followed by 100° C. for 1 min. The reaction vial was cooled to 40° C. and 2.0 mg of 2,4-dinitrobenzonitrile in 1 mL DMSO was added. The reaction vessel was then sealed and stirred for 6 min after which the mixture was diluted with 8 mL of H₂O before being loaded onto conditioned Oasis HLB and SepPak light C18 cartridges in series. The cartridges are then back flushed with 3 mL MeCN to elute the desired 2-[¹⁸F]fluoro-4-nitrobenzonitrile, which was returned to the original reaction vessel. The cartridges were eluted with 8 mL of H₂O so that they could be reused after the second reaction. The MeCN was removed under a stream of nitrogen gas at 60° C. for 5 min, and 1 mL of 2 M potassium hydroxide was added to the residue. The reaction vessel was then sealed and heated to 105° C. for 10 min, and then cooled to 40° C. before 2 mL of 2 M acetic acid and 5 mL of H₂O were added. The mixture was stirred before being passed over previously used HLB and C18 cartridges. The cartridges were then back flushed with 1.5 mL MeCN to elute 2-[¹⁸F]fluoro-4-nitrobenzoic acid to a second reaction vessel, and the MeCN was removed under a stream of nitrogen at 60° C. Ethyl tosylate (50 μg) in 1.0 mL DMF/saturated Na₂CO₃(aq) was then added to the second reaction vessel, which was then sealed and heated to 105° C. for 5 min. After cooling, 4.5 mL of H₂O was added to the reaction vessel, and the resulting mixture was then loaded onto a semipreparative HPLC column (Phenomenex Luna C18(2), 10 μm, 250×10 mm) and eluted with 45% MeCN/55% H₂O at a flow rate of 5 mL/min. The product fraction was collected and diluted with H₂O (30 mL) and then passed through a second C18 Light SepPak cartridge. The final product was eluted from the SepPak with ethanol (1.0 mL) into a product vial precharged with USP saline (10 mL). Finally, the formulated product solution was passed through a membrane filter (Millipore Millex GV, 0.22 μm) into a vented sterile empty vial. A portion of the product solution was then taken for quality control tests as performed in the manual radiosynthesis procedure.

### EXAMPLE 2

### Radiosynthesis of 2-[¹⁸F]F-NB

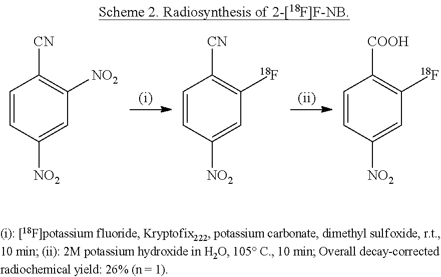 [SMILES: N#Cc1ccc([N+](=O)[O-])cc1[N+](=O)[O-] ; N#Cc1ccc([N+](=O)[O-])cc1[18F] ; O=C(O)c1ccc([N+](=O)[O-])cc1[18F]]

Automated Radiosynthesis of 2-[¹⁸F]F-4-Nitrobenzoate (2-[¹⁸F]F-NB). 2-[¹⁸F]F-NB was synthesized using a GE Tracerlab FXN pro radiosynthesis box. [¹⁸F]Fluoride was produced at an offsite cyclotron and arrived at our facility around 4 h EOB. The [¹⁸F] fluoride was trapped on a Waters SepPak light Accell plus QMA cartridge and then eluted with 1 mL of 96% MeCN containing potassium carbonate (4 mg/mL)/Kryptofix K_{2.2.2} (14.4 mg/mL) into the reaction vessel. This solution was evaporated to dryness under a stream of nitrogen at 70° C. for 7 min followed by 100° C. for 1 min. The reaction vial was cooled to 40° C. and 2.0 mg of 2,4-dinitrobenzonitrile in 1 mL DMSO was added. The reaction vessel was sealed and stirred for 6 min after which the mixture was diluted with 8 mL of H₂O before being loaded onto conditioned Oasis HLB and SepPak light C18 cartridges in series. The cartridges were then back flushed with 3 mL MeCN to elute the desired 2-[¹⁸F]fluoro-4-nitrobenzonitrile, which was returned to the original reaction vessel. The cartridges were then eluted with 8 mL of H₂O so that they could be reused after the second reaction. The MeCN was removed under a stream of nitrogen gas at 60° C. for 5 min, and 1 mL of 2 M potassium hydroxide was added to the residue. The reaction vessel was sealed and heated to 105° C. for 10 min after which it was then cooled to 40° C. before 2 mL of 2 M acetic acid and 2 mL of H₂O were added. The mixture was loaded onto a semipreparative HPLC column (Phenomenex Luna C18(2), 10 μ, 250×10 mm) and eluted with 25% MeCN containing 0.1% TFA at a flow rate of 5 mL/min. The product fraction (eluting at 14-16 min) was collected and diluted with H₂O (30 mL). The diluted product solution was passed through a second HLB SepPak cartridge. The final product, 2-[¹⁸F] fluoro-4-nitrobenzoic acid, was eluted from the SepPak with ethanol (1.0 mL) into a product vial precharged with USP saline (10 mL). Finally, the formulated product solution was passed through a membrane filter (Millipore Millex GV, 0.22 μm) into a vented sterile empty vial. A portion of the product solution was then taken for quality control tests as described in the manual radiosynthesis procedure.

### EXAMPLE 3

### Synthesis of Ethyl 2-Fluoro-4-nitrobenzoate (F-ENB)

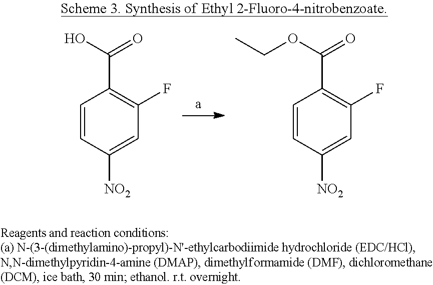

Synthesis of Ethyl 2-Fluoro-4-nitrobenzoate (F-ENB). Ethyl 2-fluoro-4-nitrobenzoate (F-ENB) was synthesized from 2-fluoro-4-nitrobenzoic acid as shown in Scheme 3. 2-Fluoro-4-nitrobenzoic acid (200 mg, 0.943 mmol), EDC/HCl (199 mg, 1.037 mmol), DMAP (23 mg, 0.188 mmol), a drop of DMF were added to a 50 mL RBF containing 15 mL DCM. The reaction mixture was stirred on ice for 30 min, after which ethanol (2 mL) was added and the mixture stirred at room temperature overnight. After the reaction was shown to be complete by TLC (30% ethyl acetate in hexane), the crude product was purified by Combiflash using a silica gel column and petroleum ether and ethyl acetate (30% ethyl acetate in hexane) as the mobile phase to yield F-ENB. ¹H NMR (400 MHz, DMSO-d₆): δ 1.33 (t, J=7.09 Hz, 3 H), δ 4.37 (q, J=7.09 Hz, 2 H), δ 8.08-8.19 (m, 2 H), δ 8.23 (dd, J=10.51, 1.71 Hz, 1 H). ¹⁹F NMR (376 MHz, DMSO-d₆): δ −106.99 (s, 1 F).

### EXAMPLE 4

### Synthesis of 2-Fluoro-4-aminoacetophenone

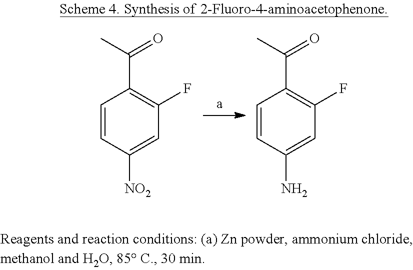

Synthesis of 2-Fluoro-4-aminoacetophenone. 2-Fluoro-4-aminoacetophenone (F-AAP) was synthesized from 2-fluoro-4-nitroacetophenone as shown in Scheme 4. 2-Fluoro-4-nitroacetophenone (50 mg, 0.27 mmol), Zn powder (178 mg, 2.73 mmol), ammonium chloride (219 mg, 4.0 mmol) were added to a 50 mL RBF containing 4.5 mL methanol and 1 mL H₂O. The reaction mixture was heated at 85° C. for 30 min under reflux. After the reaction was shown to be complete by TLC (20% ethyl acetate in hexane), the reaction mixture was cooled to room temperature and the solvent was removed and the solid crude was resuspended in H₂O and extracted with ethyl acetate. The crude product was purified by Combiflash using a silica gel column and petroleum ether and ethyl acetate (20% ethyl acetate in hexane) as the mobile phase to yield F-AAP. ESI-MS calculated for molecular ion C₈H₉FNO ([M+H]⁺): m/z=154, found m/z=154 in positive mode. ¹H NMR (400 MHz, CDCl₃-d): δ 2.56 (d, J=5.38 Hz, 3 H), 4.21 (br. s., 2 H), 6.31 (dd, J=13.20, 2.20 Hz, 1 H), 6.44 (dd, J=8.56, 2.20 Hz, 1 H), 7.77 (t, J=8.56 Hz, 1 H).

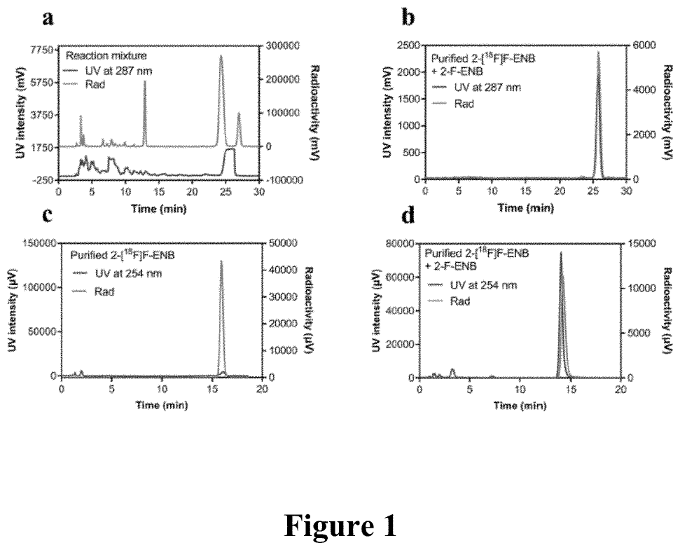

Ethyl 2-[¹⁸F]F-4-nitrobenzoate (2-[¹⁸F]F-ENB) was prepared via one-step manual radiosynthesis using ethyl 2,4-dinitro benzoate as the starting material (Scheme 1A). The radioactive product was collected following HPLC and the identity of the radiotracer was confirmed by demonstrating that the retention time of the labeled material matched that of the cold reference standard ethyl 2-F-4-nitrobenzoate (2-F-ENB) (FIG. 1a,b). The overall synthesis time was 90 min with a radiochemical yield (RCY) of 7% (n=8, decay-corrected). A primary factor in the moderate RCY of 2-[¹⁸F]F-ENB was assumed to arise from the steric hindrance of the ortho ethyl ester group, which directed the radio-fluorination preferentially to the para-position. The specific activity of 2-[¹⁸F]F-ENB was calculated to be 3983±1704 mCi/μmole using a standard curve based on the UV absorbance of 2-F-ENB at 287 nm, and the radiochemical purity was determined to be 99.2±0.7%. To improve the RCY of 2-[¹⁸F]F-ENB and facilitate clinical translation, an automated three-step radiosynthesis method using 2,4-dinitro benzonitrile as an alternative precursor was also developed (Scheme 1B and FIG. 1c,d). The total synthesis time of 2-[¹⁸F]F-ENB was 90 min with a typical decay-corrected yield of 29%, and the final product had an average specific activity of 2556 mCi/μmole.

Study Design. All protocols were approved by the Stony Brook Biosafety, Radiation Safety, and Animal Care and Use Committees, and all in vivo procedures were conducted under an IACUC approved protocol in compliance with the Animal Welfare Act and the Public Health Service Policy on Humane Care and Use of Laboratory Animals.

Bacterial Strains. Experiments utilized S. aureus Newman (ATCC 12600) or a bioluminescent strain of S. aureus (Xen29), which contains a chromosomal copy of the modified Photorhabdus luminescens luxABCDE operon at a single integration site on the bacterial chromosome. S. aureus Xen29 was obtained from PerkinElmer.

### EXAMPLE 5

### In Vitro Cellular Uptake of Radiotracer

S. aureus Newman and S. aureus Xen29 were aerobically grown to OD600 0.6 in LB media at 37° C. To evaluate the uptake of 2-[¹⁸F]F-NB, the bacteria were incubated with 1 μCi/mL of the radiotracer at 37° C. with rapid agitation. Heat-killed (90° C. for 30 min) bacteria were similarly incubated with 2-[¹⁸F]FNB. Aliquots (1 mL) were removed after 5, 30, 60, 90, and 120 min and pelleted by centrifugation (11 000 rpm, 3 min) and washed three times with cold phosphate buffered saline (PBS). Total radioactivity was measured using an automated gamma counter and is reported as the percentage of radioactivity added to the culture. A minimum of five replicates were used for each assay and time point.

### EXAMPLE 6

### Rat Triceps Infection

S. aureus (Newman or Xen29) was cultured overnight by inoculating 5 μL of bacteria from a glycerol stock into 10 mL of fresh cation-adjusted Mueller-Hinton (CAMH) broth or tryptic soy broth (TSB). 100 μL of the overnight culture was then used to inoculate 5 mL of fresh CAMH media or TSB media, which was shaken at 37° C. for about 2 h until the culture reached an OD of 0.6. A 0.5 mL sample of the culture was subsequently centrifuged at 11 000 rpm for 3 min and the cell pellet was washed twice with 0.5 mL brain heart infusion (BHI) media before being resuspended in 0.15 mL BHI media. Healthy female Sprague-Dawley rats (10 weeks old, Charles River) were intramuscularly infected with S. aureus Newman or Xen29 in the right triceps with a bacterial burden of 8 log₁₀ CFU (50 μL BHI culture). The rats were imaged by PET/CT 22 to 24 h after infection. After imaging, the infected triceps were collected, homogenized in PBS using a hand-held homogenizer, and the bacterial burden at the time of imaging was determined by plating the muscle tissues onto solid MH medium. After overnight incubation at 37° C., CFUs were quantified by enumeration. For histological examination, tissue samples were also obtained from some of the infected animals, fixed overnight in 4% formaldehyde before being embedded in paraffin, sectioned at 4 μm, and stained with hematoxylin and eosin (H&E) or Gram stain.

### EXAMPLE 7

### Sterile Inflammation in the Rat Triceps

S. aureus (Newman or Xen29) was cultured as described above except that an additional culture was autoclaved to generate a sample of heat-killed bacteria. 1 mL of the autoclaved culture was centrifuged at 11 000 rpm for 3 min, and the cell pellet was washed twice with 1 mL BHI and then resuspended in 0.15 mL BHI. Female Sprague-Dawley rats were injected with 50 μL of the heat killed bacterial culture to induce inflammation in the left triceps. To confirm the absence of live bacterial cells present in the left triceps, the triceps were collected after imaging, homogenized and plated for CFU counting. A histological examination was also conducted as described above after imaging to confirm the presence of inflammation.

### EXAMPLE 8

### PET/CT Imaging

2-[¹⁸F]F-ENB or 2-[¹⁸F]F-NB (600 to 1000 μCi) was injected via a tail vein catheter into rats that were anesthetized using 2% isoflurane with oxygen. Immediately after tracer administration, a 120 min dynamic PET scan was performed, followed by an 8 min full-body CT scan using a SIEMENS Inveon Docked PET/SPECT/CT. The CT data were reconstructed in real-time in voxel numbers that was automatically calculated by the software. After the imaging, the PET data were histogrammed into designated time frames, followed by reconstruction into PET images using the OSEM 3D method. The images were subsequently analyzed by Amide version 1.0.4 (http://www.amide.sourceforge.net).

After the 120 min dynamic scan was complete, the rats were returned to their cages for 1 h to allow them to recover from anesthesia. Three h after tracer injection, a 20 min static PET scan was performed followed by an 8 min full-body CT scan. The image data were reconstructed and analyzed in the same way as 120 min dynamic scan.

After completion of the whole-body scan, the rats were euthanized by CO₂ inhalation and the infected and inflamed triceps were excised and subsequently imaged with PET/CT scanner for 20 min. The image data were analyzed with Amide. The rest of the major organs were dissected and collected for ex vivo gamma counting described below.

For [¹⁸F]FDG PET imaging, rats were fasted 12 h before they were injected with 800 μCi of radiotracer via the tail vein. The animals were allowed to wake up between [¹⁸F]FDG injection and imaging, and PET scanning was performed 1 h after tracer administration using a 20 min list mode acquisition. The image data were reconstructed and analyzed in the same way as described above.

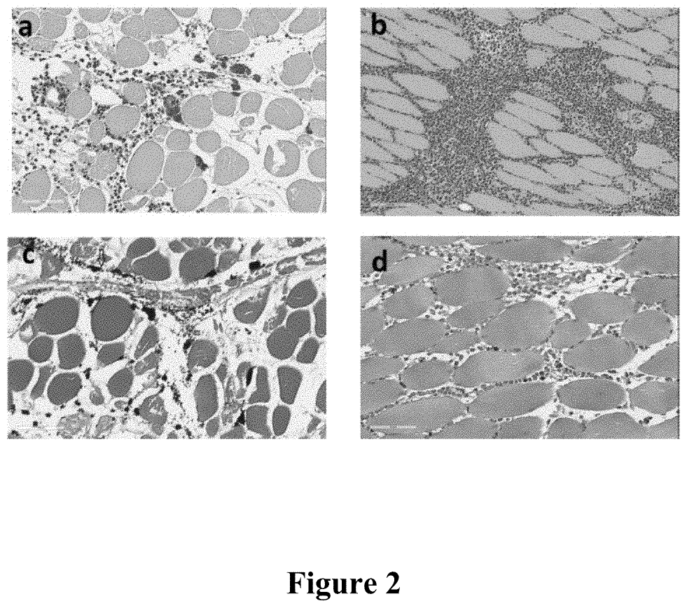

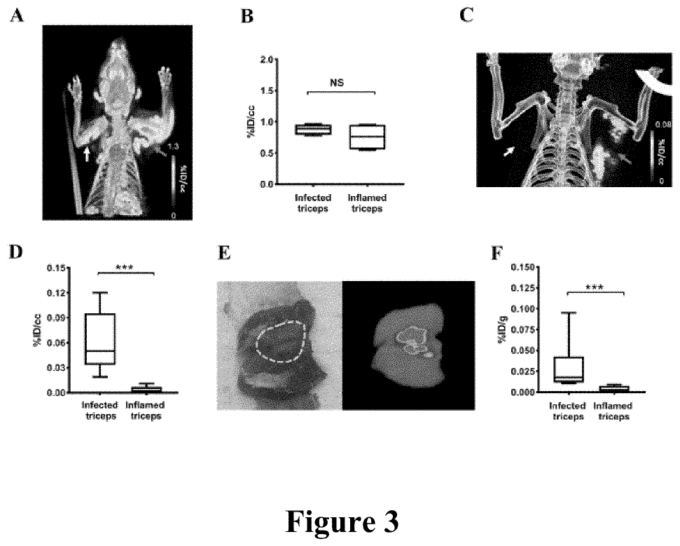

2-[¹⁸F]F-ENB PET Can Distinguish Infection from Inflammation. The critical requirement for an ideal bacteria-specific tracer is its ability to distinguish active infection from sterile inflammation. Therefore, a mixed infection and inflammation rat triceps model was used to evaluate 2-[¹⁸F]F-ENB. The model was established by intramuscular injection of S. aureus into the right triceps and a 10-fold higher burden of heat-killed S. aureus into the left triceps to induce sterile inflammation. Tissue histology confirmed the presence of S. aureus in the infected triceps while no bacterial colonies were found in the contralateral triceps (FIG. 2). The level of inflammation in both triceps was evaluated by [¹⁸F]FDG, which is commonly used to assess the host inflammatory response. The radioactivity associated with [¹⁸F]FDG in the infected and the inflamed triceps was similar (0.8% ID/cc and 1.2% ID/cc, respectively, p>0.05) (FIG. 3A,B). The biodistribution of 2-[¹⁸F]F-ENB was evaluated in this same model at 3 h post tracer administration showing that the ratio of radioactivity in the infected triceps to inflamed triceps was 17 (FIG. 3C,D).

To further examine the biodistribution of the radiotracer in the infected and inflamed triceps, both triceps were excised following in vivo PET imaging and analyzed using ex vivo tissue PET imaging, bacterial culture and histology (FIGS. 3E,F, 2 and 4). This showed that the radioactive signal in the infected triceps colocalized with regions of S. aureus Newman infection.

### EXAMPLE 9

### Bioluminescence Imaging

Upon completion of PET/CT imaging of excised infected and inflamed triceps, the same triceps were imaged with the IVIS Lumina II imaging station. Image data collection was performed using a field of view of 8 or 10 cm, exposure time of 5 min, binning number of 16, and an f1/stop of 1. The image analysis was performed with the Living Image software package. The signal intensity of the region of interest (ROI) is represented by radiance (p/sec/cm²/sr), which refers to the number of photons per second that are leaving a square centimeter of tissue and radiating into a solid angle of one steradian (sr).

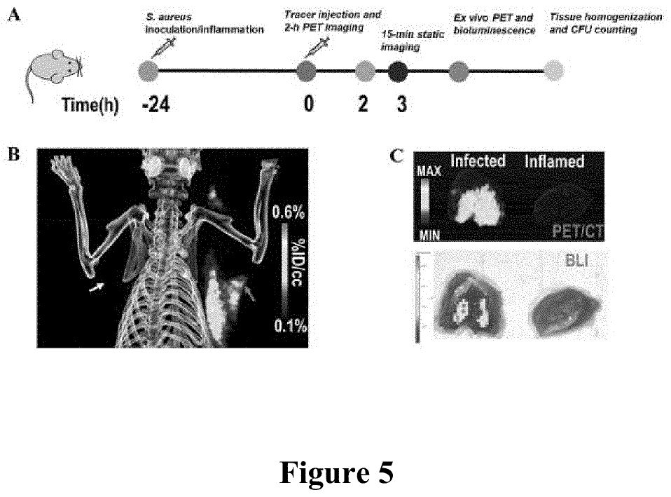

2-[¹⁸F]F-ENB-Associated Radioactivity Colocalizes with S. aureus Bioluminescence. The bioluminescent S. aureus strain Xen29 has previously been used to monitor antibiotic efficacy using preclinical bioluminescence imaging (Xiong, Y. Q. et al. 2005; Hertlein, T. et al. 2014) and also as a complementary in vivo imaging modality (van Oosten, M. et al. 2013). Therefore, to confirm that the PET signal in the infected triceps was derived from S. aureus, the triceps infection was induced using S. aureus Xen29 for colocalization experiments. At 180 min post tracer injection, the radioactive signal was predominantly observed in the infected triceps, consistent with the imaging data for the Newman strain of S. aureus (FIG. 5B). Ex vivo PET imaging further demonstrated the localization of radioactivity in the infected triceps, while ex vivo bioluminescence showed colocalization of the PET signal with signal arising from bioluminescence imaging (BLI) (FIG. 5C).

### EXAMPLE 10

### Ex Vivo Radiotracer Biodistribution

The biodistribution study was performed after the completion of ex vivo tissue PET/CT imaging and bioluminescence imaging described. The major organs and tissues were collected, weighed, and placed in gamma counter tubes. The organ-associated radioactivity was measured using a gamma counter (Wizard 2480, PerkinElmer) and is expressed as % ID/g (percentage of injected dose per gram of organ).

### EXAMPLE 11

### In Vitro Metabolic Stability of the Radiotracers in Rat Plasma

2-[¹⁸F]F-ENB (600 μCi) was incubated with Sprague-Dawley rat plasma (5.0 mL) at 37° C. Subsequently, 0.4 mL aliquots of the plasma were taken 2, 10, 30, and 85 min after addition of the tracer, and 0.6 mL of MeCN was added to quench the metabolism. The mixture was centrifuged at 13 400 rpm for 10 min to remove proteins, and both the supernatant and the protein pellet were analyzed by gamma counting to calculate the extraction efficiency. The supernatant was spiked with 50 μL cold standards (F-PABA, 2-fluoro-4-nitrobenzoic acid, ethyl 2-fluoro-4-aminobenzoate, ethyl 2-fluoro-4-nitrobenzoate, 1 mg/mL) and injected onto a semiprep HPLC column (Phenomenex Luna C18(2), 10 μm, 250×10 mm) that was then eluted with 45% MeCN/55% H₂O, 0.1% trifluoroacetic acid, at a flow rate of 4 mL/min. The radioactive fractions corresponding to each cold standard were collected and radioactivity was quantified using a gamma counter from which the percentage of each metabolite was calculated.

### EXAMPLE 12

### In Vitro Metabolic Stability of the Radiotracers in Human Plasma

2-[¹⁸F]F-ENB (600 μCi) was incubated with 5.0 mL human serum (catalog #: H4522 from Sigma) at 37° C. Subsequently, 0.4 mL aliquots of the plasma were taken 2, 10, 30, and 85 min after addition of the tracer, and 0.6 mL of MeCN was added to quench the metabolism. The mixture was centrifuged at 13 400 rpm for 10 min to remove proteins, and both the supernatant and the protein pellet were analyzed by gamma counting to calculate the extraction efficiency. The supernatant was spiked with 50 μL cold standards (F-PABA, 2-fluoro-4-nitrobenzoic acid, ethyl 2-fluoro-4-aminobenzoate, ethyl 2-fluoro-4-nitrobenzoate, 1 mg/mL) and injected onto a semiprep HPLC column (Phenomenex Luna C18(2), 10 μm, 250×10 mm) that was then eluted with 45% MeCN/55% H₂O, 0.1% trifluoroacetic acid, at a flow rate of 4 mL/min. The radioactive fractions corresponding to each cold standard were collected and the radioactivity was counted using a gamma counter from which the percentage of each metabolite was calculated (n=3).

### EXAMPLE 13

### In Vivo Metabolite Analysis

2-[¹⁸F]F-ENB (1 mCi) was injected via the tail vein into healthy female Sprague-Dawley rats (10 weeks old, Charles River). At 5, 30, and 60 min (n=3 rats at each time point), 1 mL of blood was obtained by cardiac puncture, centrifuged, and 50 μL of each standard (including N-acetyl F-PABA, F-PABA, 2-fluoro-4-nitrobenzoic acid, ethyl 2-fluoro-4-aminobenzoate and ethyl 2-fluoro-4-nitrobenzoate, 1 mg/mL) was added to 0.4 mL aliquots of the resulting supernatant together with 0.6 mL MeCN. The mixture was centrifuged at 13 400 rpm for 5 min to remove the protein, and the supernatant and protein pellet were analyzed by gamma counter to determine the efficiency of extraction. The supernatant was injected onto a semiprep HPLC column (Phenomenex Luna C18(2), 10 μm, 250×10 mm) and chromatography was performed with 45% MeCN/55% H₂O containing 0.1% trifluoroacetic acid, at a flow rate of 4 mL/min. The radioactive fractions corresponding to each cold standard were collected and analyzed using a gamma counter from which the percentage of each metabolite was calculated (n=3).

Analysis of the radioactive metabolites in the urine was performed using a similar procedure from samples obtained from the bladder 30 and 60 min after tracer injection. The urine was diluted with 45% MeCN/H₂O, centrifuged at 13 400 rpm for 5 min and spiked with cold standards for radio-HPLC analysis.

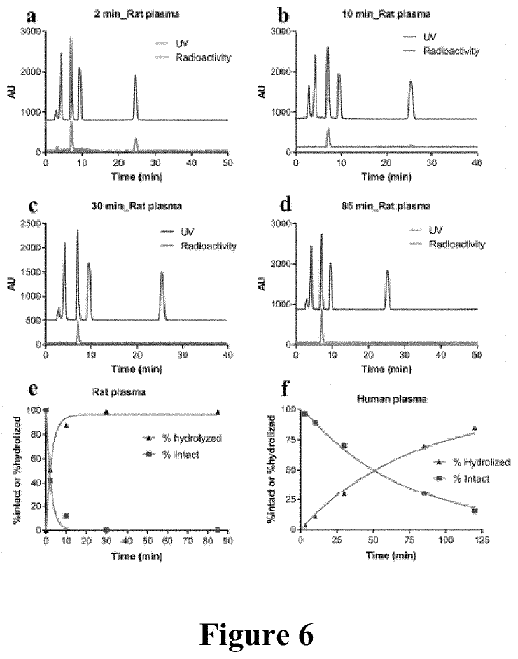

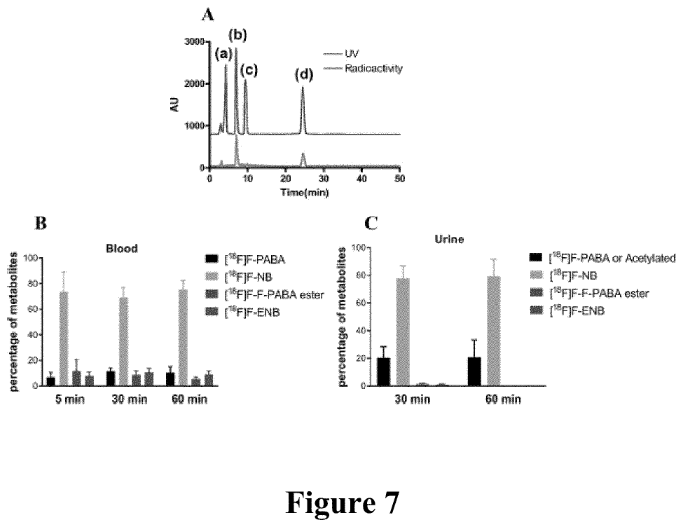

2-[¹⁸F]-ENB Is Rapidly Hydrolyzed to 2-[¹⁸F]F-NB but the Nitro Group Is Metabolically Stable. The in vitro metabolic stability of 2-[¹⁸F]F-ENB was determined to assess the lability of the ester protecting group and the overall stability of the nitro group. We found that 2-[¹⁸F]F-ENB was efficiently hydrolyzed in rat serum to the corresponding acid 2-[¹⁸F]fluoro-4-nitrobenzoate (2-[¹⁸F]F-NB) with a half-life of 2 min whereas the nitro group was unaffected over the 85 min incubation period (FIG. 6a-e). A similar result was observed in human serum although the half-life for hydrolysis was 35 min (FIG. 6f). We then analyzed the stability of 2-[¹⁸F]F-ENB in rat blood in vivo. After intravenous administration of 2-[¹⁸F]F-ENB into healthy rats, blood samples were taken at 5, 30, and 60 min and analyzed by radio-HPLC, which demonstrated that 2-[¹⁸F]F-NB was the predominant radioactive component at each time point (FIG. 7B). Analysis of urine samples at 30 and 60 min post injection also revealed that the majority of the radioactive signal could be attributed to 2-[¹⁸F]F-NB (FIG. 7C). Together, these data indicate that the ester is rapidly hydrolyzed but that the aromatic nitro group of 2-[¹⁸F]F-ENB is metabolically stable in vivo. The in vivo studies also revealed small amounts of 2-[¹⁸F]F-PABA and the N-acetylated analogue of 2-[¹⁸F]F-PABA in the blood and urine, and it is noted that both the liver and bacteria in the intestinal flora contain nitroreductases capable of reducing aromatic nitro groups (Zachariah, P. K. & Juchau, M. R. 1974; Wheeler, L. A. et al. 1975). On the basis of studies with 4-nitrobenzoic acid, it is likely that intestinal bacteria are the primary source of the nitroreductase activity (Zachariah, P. K. & Juchau, M. R. 1974; Wheeler, L. A. et al. 1975).

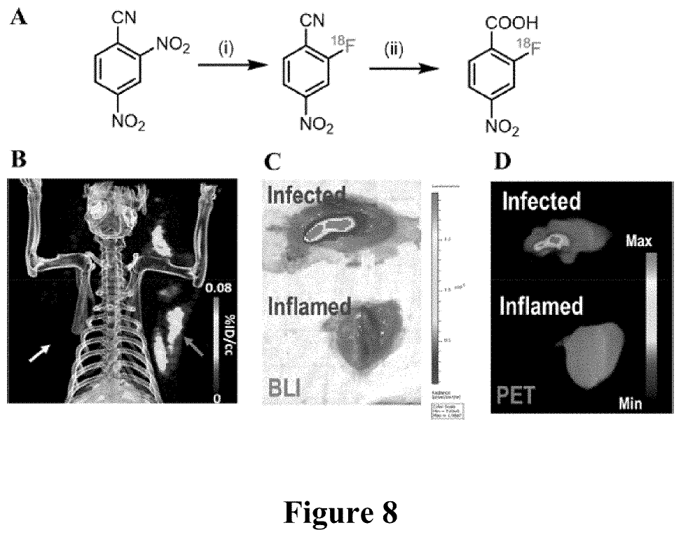

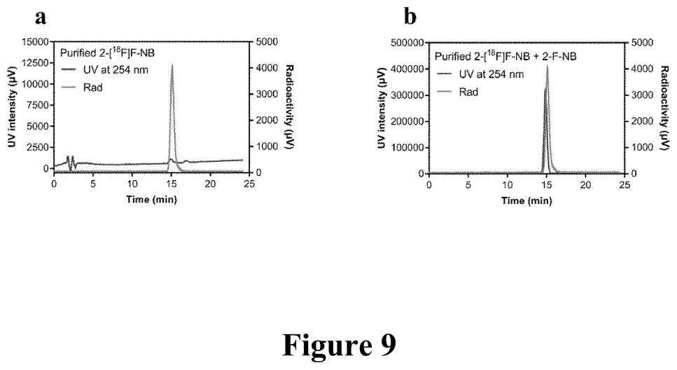

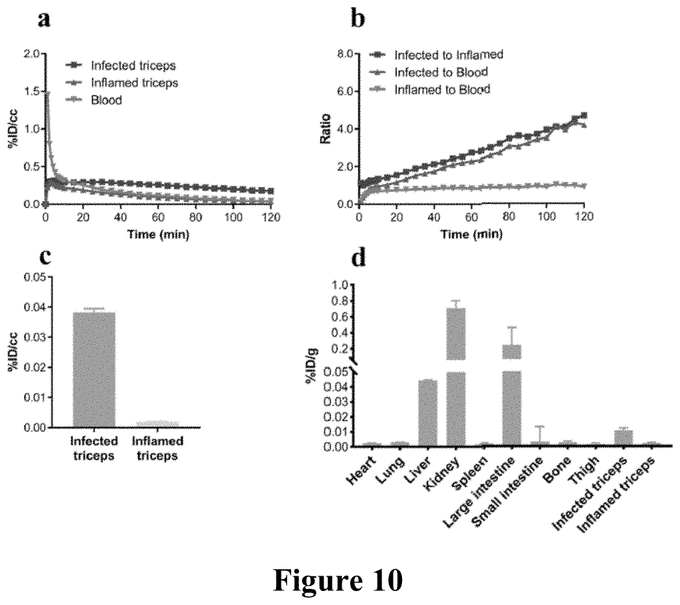

2-[¹⁸F]F-NB Is Responsible for the Radioactive Signal at the Site of Infection. Due to the rapid hydrolysis of 2-[¹⁸F]F-ENB to 2-[¹⁸F]F-NB in plasma, we hypothesized that 2-[¹⁸F]F-NB would be the active radiotracer responsible for the PET signal at the site of infection. To test this hypothesis, 2-[¹⁸F]F-NB was radiosynthesized using a GE Tracerlab FXN Pro Radiosynthesis Module (FIGS. 8A and 9) and evaluated in the same infection model. At 3 h post injection, PET imaging results demonstrated that the radioactivity associated with 2-[¹⁸F]F-NB was localized at the site of infection and also colocalized with the bioluminescence signal arising from S. aureus Xen29 (FIG. 8B-D). Quantitative analysis of the ROI showed that the signal at the infection site was 17-fold higher than the one at the site of sterile inflammation, an observation supported by ex vivo gamma counting (FIG. 10c,d).

### EXAMPLE 14

### The Nitroreductase Assay

NfsB was expressed and purified as described previously (Si, Y. et al. 2019). The nitroreductase assay was performed at room temperature in 50 mM Tris-HCl buffer, pH 7.4, containing 5 mM EDTA in a total reaction volume of 500 μL. Reactions were initiated by the addition of NfsB to a final concentration of 45 nM, and the consumption of NADH was continuously monitored at 340 nm. The k_{cat} and Kₘ values were determined at a fixed concentration of NADH (60 μM NADH for NfsB) by varying the concentration of 2-FENB, 2-F-NB, or 2-F-NAP. The experiments were performed in triplicate and the initial velocities as a function of substrate concentration were globally fit to the Michaelis-Menten equation using GraphPad Prism 4.

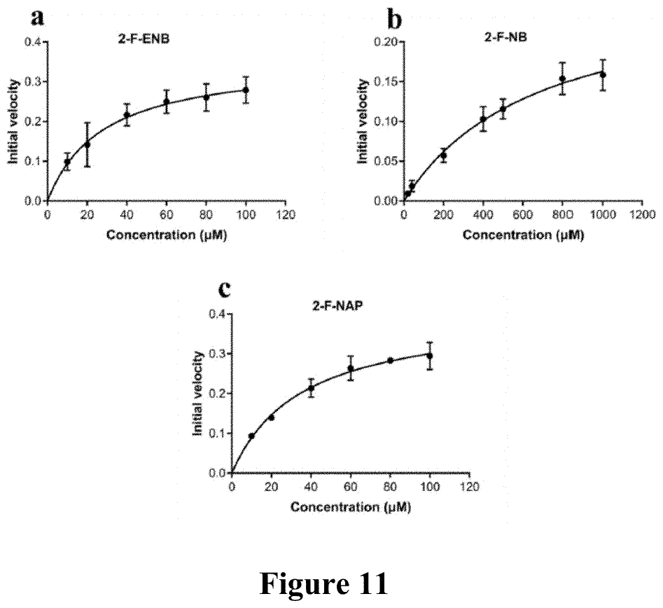

2-F-ENB and 2-F-NB Are Substrates for S. aureus NfsB. Above we have shown that 2-[¹⁸F]F-ENB is rapidly hydrolyzed to 2-[¹⁸F]F-NB, and that 2-[¹⁸F]F-NB recapitulates the imaging results obtained with 2-[¹⁸F]F-ENB. We next assessed the ability of the aromatic nitro group in 2-F-ENB and 2-F-NB to be reduced to the corresponding amine by the S. aureus-specific nitroreductase NfsB. S. aureus NfsB was cloned, expressed and purified, and F-ENB and F-NB were evaluated as substrates using NADH as the cofactor (Si, Y. et al. 2019). NfsB was found to efficiently reduce both 2-F-ENB and 2-F-NB, with k_{cat}/Kₘ values of 5.7×10⁵ M⁻¹ s⁻¹ and 1.8×10⁴ M⁻¹ s⁻¹, respectively (Table 1 and FIG. 11). This differential activity of S. aureus NfsB toward the ester and acid was also observed for both the Escherichia coli nitroreductases NfsA and NfsB toward methyl 4-nitrobenzoate and 4-nitrobenzoate (Zenno, S. et al. 1996). Thus, the data support the proposal that the in vivo signal at the site of infection could result from the conversion of the radiotracers into 2-[¹⁸F]F-PABA, which is a substrate for DHPS.

TABLE 1
Reduction of F-ENB and F-NB by NfsB^{a}

| substrate | k_{cat} (s⁻¹) | Kₘ (μM) | k_{cat}/Kₘ (M⁻¹ s⁻¹) |
|---|---|---|---|
| 2-F-ENB | 16.1 ± 1.7 | 28 ± 8 | 5.7 × 10⁵ |
| 2-F-NB | 12.4 ± 1.6 | 686 ± 174 | 1.8 × 10⁴ |

^{a}Initial velocities were monitored at 340 nm at a fixed concentration of NADH (40 μM) and at varying concentrations of 2-F-ENB and 2-F-NB. Data were fit to the Michaelis-Menten equation.

### EXAMPLE 15

### Minimum Inhibitory Concentration (MIC)

Antibacterial susceptibility tests for aerobically growing bacteria were performed with a modified microbroth dilution assay according to the Clinical and Laboratory Standard Institute in transparent 96-well plates (CLSI. 2006). S. aureus Xen29 (PerkinElmer) were utilized in this study. Briefly, bacteria were grown to mid log phase (OD600 of 0.6-0.7) in cation-adjusted Mueller-Hinton (CAMH) media at 37° C. in an orbital shaker. Subsequently, bacteria were resuspended in the M9 media. A final concentration of 10⁶ CFU/mL per well was added to M9 media (1% glucose, 1% casamino acids, 1 mM thiamine, 0.05 mM nicotinamide, 2 mM MgCl₂, 0.1 mM CaCl₂) containing 2-fold dilutions of SMX to give final concentrations ranging from 0.05 to 100 μg/mL either alone or in the presence of PABA (0.8 μg/mL and 4 μg/mL), F-PABA (0.8 μg/mL and 4 μg/mL), F-NB (10 μg/mL, 20 μg/mL and 40 μg/mL), or F-NAP (10 μg/mL, 20 μg/mL and 40 μg/mL). The plate was examined for bacteria growth using a UV-vis plate reader after 24 h incubation at 37° C. The MIC was recorded as the lowest SMX concentration (μM) required to inhibit at least 90% of bacterial growth as judged by the absorbance of the culture media.

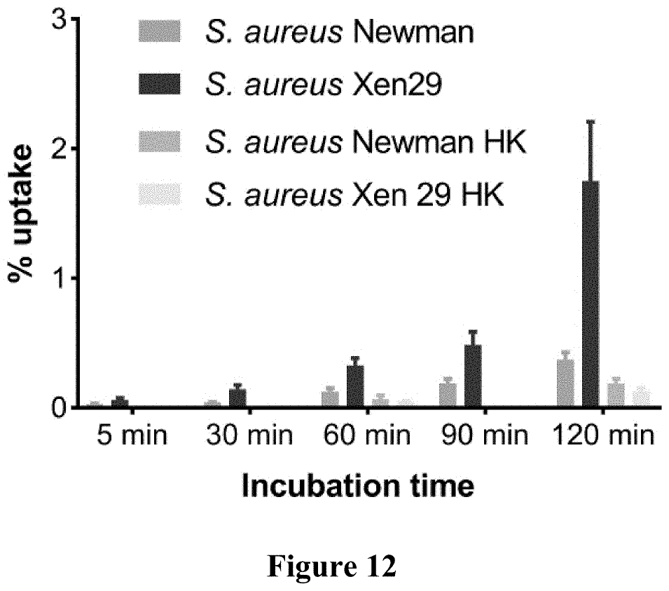

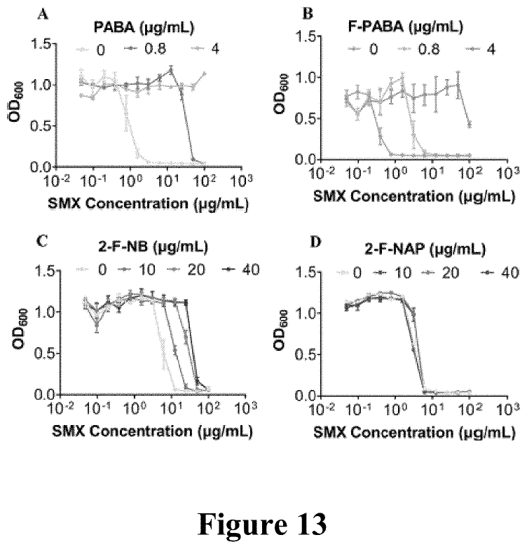

2-F-NB Can Antagonize the Bacteriostatic Effect of Sulfonamide with S. aureus Xen29. F-PABA is a substrate for dihydropteroate synthase (DHPS) in the folate biosynthesis pathway, and to provide further evidence for the proposed mechanism of action (MOA) of our second generation tracers, we established a phenotypic assay to evaluate the ability of F-PABA and F-NB to rescue the antibacterial effect of the DHPS inhibitor sulfamethoxazole (SMX). We first demonstrated that 2-[¹⁸F]F-NB was taken up by both S. aureus Newman and Xen29. Time dependent uptake of 2-[¹⁸F]F-NB was observed during the 120 min incubation period but not by heat killed bacteria, suggesting a specific uptake mechanism (FIG. 12). We then validated the MOA assay with the endogenous DHPS substrate PABA, and showed that bacterial regrowth in the presence of sulfamethoxazole occurred upon the addition of PABA (FIG. 13A). F-PABA also rescued bacterial growth in a concentration-dependent manner and it was shown that 0.8 and 4 μg/mL of F-PABA increased the minimum inhibitory concentration (MIC) of sulfamethoxazole by 8-fold (1.56 to 12.5 μg/mL) and to over 100 μg/mL, respectively (FIG. 13B). Similarly, 2-F-NB also antagonized the activity of sulfamethoxazole, and 40 μg/mL 2-F-NB resulted in a shift in the sulfamethoxazole MIC from 1.56 to 12.5 μg/mL (FIG. 13C). In contrast, 2-fluoro-4-nitroacetophenone (2-F-NAP), a structural analogue of FNB and also an efficient substrate for the S. aureus nitroreductase NfsB (k_{cat}/Kₘ=5.7×10⁵ M⁻¹ s⁻¹, FIG. 11), was unable to rescue growth in the presence of sulfamethoxazole (FIG. 6D). Furthermore, our previously established HPPK-DHPSDHFR coupled assay demonstrated that 2-fluoro-4-aminoacetophenone was not an efficient substrate for DHPS even at a concentration of 80 μM, supporting the link between the folate pathway and the MOA of 2-F-NB.

### EXAMPLE 16

### The Radiotracer 2-[¹⁸F]F-ENB is Able to Localize and Detect Infective Endocarditis in a Rat Model

Female Sprague-Dawley rats (weight, ˜200 g, Charles River) were maintained in an anesthetized state with an isofluorane-oxygen gas mixture during surgery. A sterile polyethylene catheter was placed and positioned in the left ventricle through a small incision via the retrograde transcarotid artery approach.

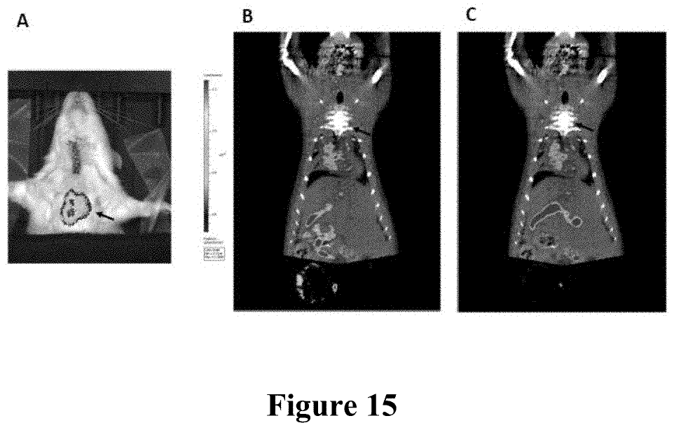

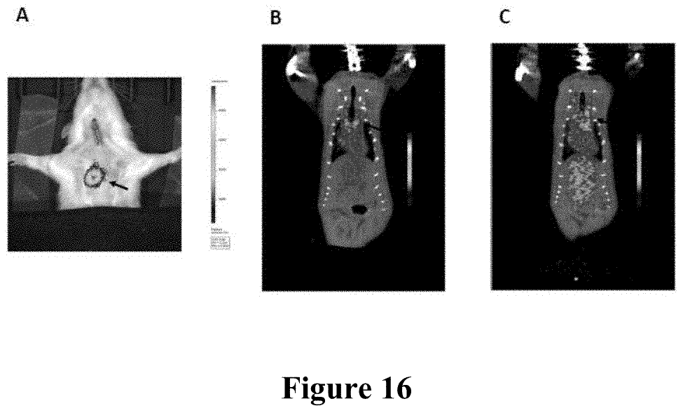

The catheter was left in place throughout the experiments. At 24-hour post-catheterization, the rats were infected intravenously through the tail vein with an inoculum of S. aureus Xen29 of 10⁶ CFU in 0.5 mL sterile saline. The animals were imaged for bioluminescence signal in the region of the hearts with IVIS daily after infection. The optimal BLI signal over the heart area was determined to be day-3 post inoculation (FIG. 15A and FIG. 16A). At day-3 post infection, a dose of [18F]F-ENB was invravaously injected to the IE rats, at 60 min (FIG. 15B and FIG. 16B) and 120 min (FIG. 15C and FIG. 16C) post tracer injection, a robust radioactive signal was detected over the heart region.

### EXAMPLE 17

### The Radiotracer 2-[¹⁸F]F-NB is Able to Localize and Detect Prosthetic Joint Infection in a Mouse Model

Upon completion of in vivo PET/CT imaging, the heart, lung and muscle were dissected and imaged with the IVIS Lumina II imaging station. Image data collection was performed using a field of view of 8 or 10 cm, exposure time of 3 min, binning number of 16, and an f1/stop of 1. The image analysis was performed with the Living Image software package. The signal intensity of the region of interest (ROI) is represented by radiance (p/sec/cm2/sr), which refers to the number of photons per second that are leaving a square centimeter of tissue and radiating into a solid angle of one steradian (sr).

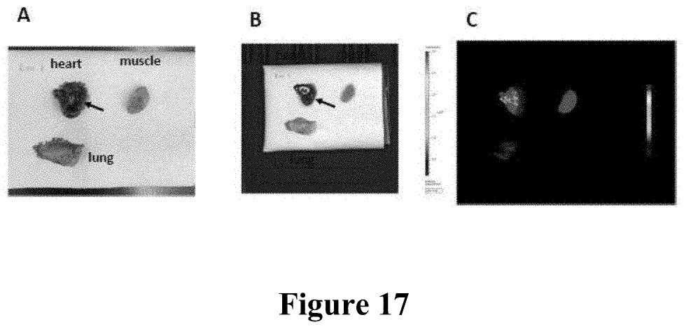

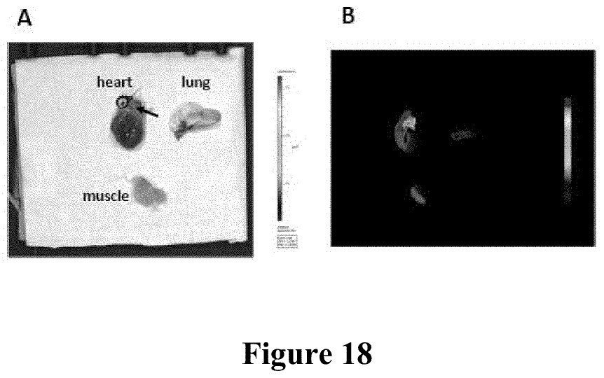

Tissue autopsy and bioluminescence Imaging showed localized BLI signal in the heart (FIG. 17A-B and FIG. 18A).

2-[¹⁸F]F-ENB-Associated Radioactivity Colocalizes with S. aureus Bioluminescence. To confirm that the in vivo PET signal was derived from infected heart, Ex vivo PET imaging of heart further demonstrated the localization of radioactivity in the infected heart (FIG. 17C and FIG. 18B).

### EXAMPLE 18

### Ex Vivo Radiotracer Biodistribution

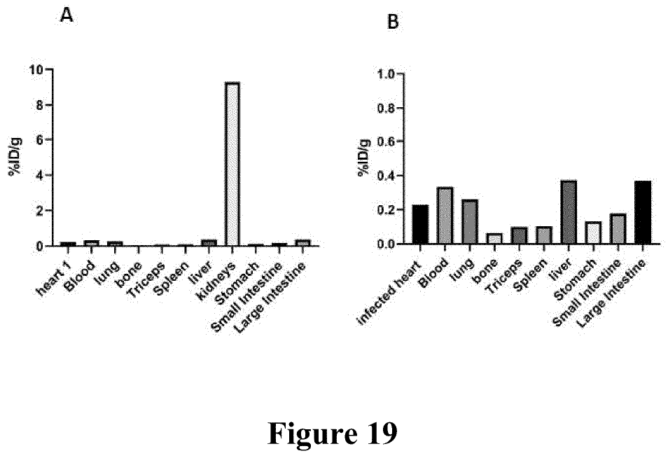

The biodistribution study was performed after the completion of ex vivo tissue PET/CT imaging and bioluminescence imaging described. The major organs and tissues were collected, weighed, and placed in gamma counter tubes. The organ-associated radioactivity was measured using a gamma counter (Wizard 2480, PerkinElmer) and is expressed as % ID/g (percentage of injected dose per gram of organ) (FIG. 19).

### EXAMPLE 19

### Development of Prosthetic Joint Infection (PJI) and PET Imaging with 2-[¹⁸F]F-ENB

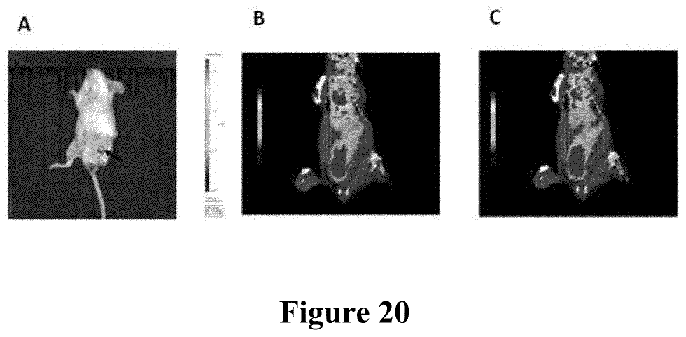

Balb/c mice was anesthetized by inhalation of a mixture of isofluorane and oxygen. A medical-grade stainless steel Kirschner wire was inserted into the distal part of the femur which has been exposed by dislocation of the patella and reamed with a 25-gauge needle. 2 μL of S. aureus Xen36 (4 log 10 CFU) will be inoculated into the knee joint and onto the wire before the surgical incision is closed with absorbable sutures. The correct position of the wire in the canal was performed with X-ray scan. The animals were imaged for bioluminescence signal in the region of the surgical joint with IVIS daily after infection. The optimal BLI signal over the joint area was determined to be day-2 post inoculation (FIG. 20A). At day-2 post infection, a dose of [¹⁸F]F-ENB was invravaously injected to the PJI rats, at 60 min (FIG. 20B) and 120 min (FIG. 20C) post tracer injection, a robust radioactive signal was detected over the joint region.

### EXAMPLE 20

### Quantitative Analysis of Time Activity Curve of PJI Mice and Ex Vivo Biodistribution

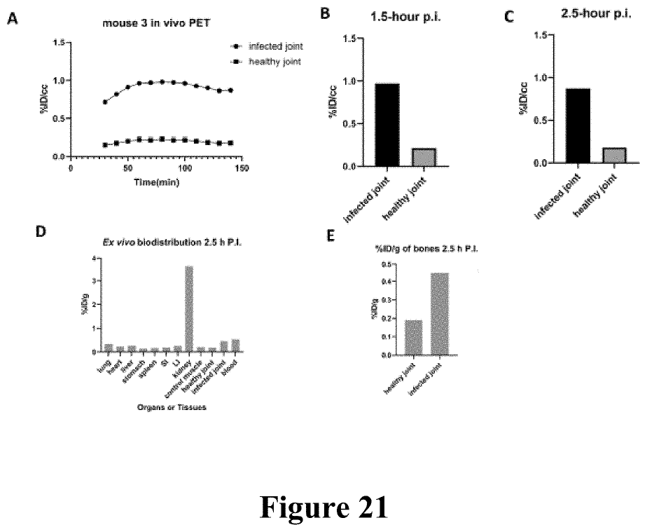

The biodistribution study was performed after the completion of ex vivo tissue PET/CT imaging and bioluminescence imaging described. The major organs and tissues were collected, weighed, and placed in gamma counter tubes. The organ-associated radioactivity was measured using a gamma counter (Wizard 2480, PerkinElmer) and is expressed as % ID/g (percentage of injected dose per gram of organ) (FIG. 21).

### EXAMPLE 21

### 2-[¹⁸F]F-ENB-Associated Radioactivity in the Infected Joint Colocalizes with S. aureus Bioluminescence

Upon completion of in vivo PET/CT imaging, both joints (infected and healthy) were dissected and imaged with the IVIS Lumina II imaging station. Image data collection was performed using a field of view of 8 or 10 cm, exposure time of 3 min, binning number of 16, and an f1/stop of 1. The image analysis was performed with the Living Image software package. The signal intensity of the region of interest (ROI) is represented by radiance (p/sec/cm2/sr), which refers to the number of photons per second that are leaving a square centimeter of tissue and radiating into a solid angle of one steradian (sr).

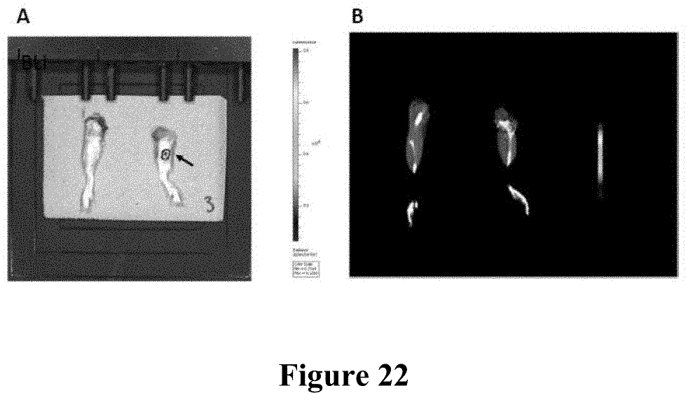

Tissue autopsy and bioluminescence Imaging showed localized BLI signal in the joint (FIG. 22A).

To confirm that the in vivo PET signal was derived from infected joint, Ex vivo PET imaging of joint further demonstrated the co-localization of radioactivity in the infected joint with bioluminescent signal emitted from S. aureus Xen 36 in the joint (FIG. 22B).

### DISCUSSION

The composition, and method of synthesizing the same, described herein contains 2-[¹⁸F]F-ENB, a PET tracer which shows high selectivity for bacteria imaging in soft-tissue rodent models of MSSA, MRSA and E. coli infection. Compared to FDG and other recently developed bacterial infection tracers, 2-[¹⁸F]F-ENB has several advantages. It is specific for bacterial infection and capable of differentiating infection from sterile inflammation. It shows high signal-to-background ratio in animal infection models. It is able to quantify bacterial burden. It can be produced with high yield using a rapid radiosynthesis method. 2-[¹⁸F]F-ENB may also be used for applications in bacterial infection diagnosis.

As a bacterial infection tracer which can potentially diagnose infections caused by a broad spectrum of pathogens including S. aureus, E. coli, Klebsiella pneumoniae, Pseudomonas aeruginosa and Staphylococcus epidermidis, 2-[¹⁸F]F-ENB has great commercialization potential. Some infectious conditions caused by S. aureus can serve as a good illustration of the commercialization potential. S. aureus is the leading cause of many different clinically important infections including skin and soft tissue infections, osteomyelitis and infectious endocarditis.

Osteomyelitis is the infectious condition of bone, which leads to inflammation and bone necrosis. With an incidence of 21.8 per 100,000 person/years in the United States, osteomyelitis is a serious infectious disease that can result in limb amputation and even death. Indeed, osteomyelitis is the leading cause of non-traumatic amputation in US and worldwide. Osteomyelitis is closely associated with diabetes, a disease that affects 7% of the world's population. Approximately 15% of diabetic patients would develop foot ulcers in their lifetime, with an annual incidence of 1 to 4%. Among the diabetic patients with foot ulcers, over 50% would be infected and develop diabetic foot infections.

Infective endocarditis (IE) is the infection of the endocardial surface of the heart. The estimated incidence rate of IE is 30 to 100 per million person-years, and S. aureus is the causative agent in over half the cases. Despite medical advances, the in-hospital mortality rate of IE is 9.6 to 26%, partly because a definitive diagnosis cannot be reached at an early stage of infection due to the lack of a sensitive diagnostic method. Currently, the diagnosis of IE depends on a combination of microbiological tests and echocardiography together with clinical signs of infection. However, none of these methods provide sufficient sensitivity or specificity to make a rapid and one-step definitive diagnosis.

Patients who are at high risk of these infections may potentially benefit from 2-[¹⁸F]F-ENB. Moreover, the potential of 2-[¹⁸F]F-ENB is not limited to S. aureus infections. Since our data has already shown that 2-[¹⁸F]F-ENB is also able to diagnose E. coli and Klebsiella pneumoniae infections, patients who are suspected of infections caused by these two bacterial species may also be candidates for 2-[¹⁸F]F-ENB. Besides, due to the existence and essentiality of folate biosynthesis pathway, which incorporates 2-[¹⁸F]F-ENB into bacterial cell components, for various bacterial species including Mycobacterium tuberculosis (M. tb), 2-[¹⁸F]F-ENB also has the commercialization potential as a diagnostic tool for such infections.

The composition and method described herein provides a fluorine-18-labeled analog of p-aminobenzoic acid (2-fluoro-4-aminobenzoic acid, F-PABA). F-PABA is a non-toxic substrate (MIC>100 μg/ml) for DHPS and is not toxic to either bacterial or mammalian cells. It has several advantages over FDG and other reported bacterial infection tracers, including:
1. 2-[¹⁸F]F-ENB is selectively taken up by live bacteria (MRSA, E. coli and M. tuberculosis) but not mammalian cells;
2. It is specific for bacterial infection, and capable of differentiating infection from inflammation;
3. It can be produced using a rapid radiosynthesis method with high radiochemical yield;
4. It accumulates in a wide range of bacteria, including E. coli, S. aureus and Klebsiella pneumomae;
5. It shows very good signal-to-background ratio in in vivo infection models; and
6. It is capable of quantifying bacterial burden, and therefore can be used to monitor drug treatment efficacy and to assist new antibacterial agent development.

### REFERENCES

Auletta, S. et al. (2019) PET Radiopharmaceuticals for Specific Bacteria Imaging: A Systematic Review. J. Clin. Med. 8, 197.

Bettegowda, C. et al. (2005) Imaging bacterial infections with radiolabeled 1-(2′-deoxy-2′-fluoro-beta-D-arabinofuranosyl)-5-iodouracil. Proc Natl Acad Sci USA 102: 1145-1150.

Censullo, A. and Vijayan, T. (2017) Using Nuclear Medicine Imaging Wisely in Diagnosing Infectious Diseases. Open Forum Infect Dis. 4, No. ofx011.

CLSI. (2006) Approved Standard M7-A5, 6th ed., Clinical and Laboratory Standards Institute, Wayne, Pa.

Gowrishankar, G. et al. (2014) Investigation of 6-^{[18F]}-Fluoromaltose as a Novel PET Tracer for Imaging Bacterial Infection. PLoS One. 9(9), e107951.

Hertlein, T. et al. (2014) Bioluminescence and 19F magnetic resonance imaging visualize the efficacy of lysostaphin alone and in combination with oxacillin against Staphylococcus aureus in murine thigh and catheter-associated infection models. Antimicrob. Agents Chemother. 58, 1630-1638.

Jamar, F. et al. (2013) EANM/SNMMI guideline for 18F-FDG use in inflammation and infection. J. Nucl. Med. 54, 647-658.

Li, Z. B. et al. (2008) The synthesis of ¹⁸F-FDS and its potential application in molecular imaging. Mol Imaging Biol. 10, 92-98.

Meller, J. et al. (2007) 18F-FDG PET and PET/CT in fever of unknown origin. J. Nucl. Med. 48, 35-45.

Mutch, C. A. et al. (2018) [(11)C]Para-Aminobenzoic Acid: A Positron Emission Tomography Tracer Targeting Bacteria-Specific Metabolism. ACS Infect. Dis. 4, 1067-1072.

Namavari, M. et al. (2015) Synthesis of ^{[18F]}-labelled Maltose Derivatives as PET Tracers for Imaging Bacterial Infection. Mol Imaging Biol. 17(2), 168-176.

Northrup, J. D. et al. (2019) Radiochemical Approaches to Imaging Bacterial Infections: Intracellular versus Extracellular Targets. Int. J. Mol. Sci. 20, 5808.

van Oosten, M. et al. (2013) Real-time in vivo imaging of invasive- and biomaterial-associated bacterial infections using fluorescently labelled vancomycin. Nat. Commun. 4, 2584.

Ordonez, A. A. and Jain, S. K. (2018) Pathogen-Specific Bacterial Imaging in Nuclear Medicine. Semin. Nucl. Med. 48, 182-194.

Sellmyer, M. A. et al. (2017) Bacterial infection imaging with [(18)F]fluoropropyl-trimethoprim. Proc. Natl. Acad. Sci. U.S.A. 114, 8372-8377.

Si, Y. et al. (2019) Antibacterial Activity and Mode of Action of a Sulfonamide-Based Class of Oxaborole Leucyl-tRNA-Synthetase Inhibitors. ACS Infect. Dis. 5, 1231-1238.

Signore, A. and Glaudemans, A. W. (2011) The molecular imaging approach to image infections and inflammation by nuclear medicine techniques. Ann Nucl. Med. 25, 681-700.

Weinstein, E. A. et al. (2014) Imaging Enterobacteriaceae infection in vivo with ¹⁸F-fluorodeoxysorbitol positron emission tomography. Sci Transl Med. 6(259), 259ra146.

Wheeler, L. A. et al. (1975) The relationship between nitro group reduction and the intestinal microflora. J. Pharmacol. Exp. Ther. 194, 135-144.

Xiong, Y. Q. et al. (2005) Real-time in vivo bioluminescent imaging for evaluating the efficacy of antibiotics in a rat Staphylococcus aureus endocarditis model. Antimicrob. Agents Chemother. 49, 380-387.

Zachariah, P. K. and Juchau, M. R. (1974) The role of gut flora in the reduction of aromatic nitro-groups. Drug Metab. Dispos. 2, 74-78.

Zenno, S. et al. (1996) Biochemical characterization of NfsA, the Escherichia coli major nitroreductase exhibiting a high amino acid sequence homology to Frp, a Vibrio harveyi flavin oxidoreductase. J. Bacteriol. 178, 4508-4514.

Zhang, Z. et al. (2018) Positron Emission Tomography Imaging with 2-[(18)F]F-p-Aminobenzoic Acid Detects Staphylococcus aureus Infections and Monitors Drug Response. ACS Infect. Dis. 4, 1635-1644.

1. A composition comprising the compound having the structure: 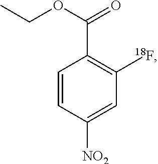
ethyl 2-[¹⁹F]F-4-nitrobenzoate, and at least one acceptable carrier.

2. The composition of claim 1, wherein the ratio of the ethyl 2-[¹⁸F]F-4-nitrobenzoate to the ethyl 2-[¹⁹F]F-4-nitrobenzoate is in a range from
a) 1:90 to 1:550;
b) 1:99 to 1:500;
c) 1:324 to 1:500; or
d) 1:99 to 1:324; or
the composition of claim 1, further comprising 4-amino-2-fluorobenzoic acid.

3-6. (canceled)

7. The composition of claim 2, wherein the ratio of the ethyl 2-[¹⁸F]F-4-nitrobenzoate to 4-amino-2-fluorobenzoic acid is about
a) 1:99;
b) 1:100;
c) 1:134; or
d) 1:500.

8-10. (canceled)

11. The composition of claim 1, wherein the radiochemical purity of ethyl 2-[¹⁸F]F-4-nitrobenzoate is at least
a) 95%;
b) 97.5%; or
c) 99%.

12-13. (canceled)

14. A process for preparing the composition of claim 1 comprising admixing at least one carrier with an amount of a compound having the structure: 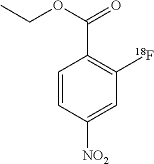
and ethyl 2-[¹⁹F]F-4-nitrobenzoate.

15. A process for preparing the compound having the structure: 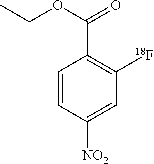
which comprises:
(a) reacting a compound having the structure: 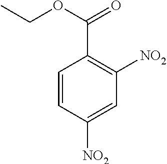 [SMILES: CCOC(=O)c1ccc([N+](=O)[O-])cc1[N+](=O)[O-]]
with a [¹⁸F] fluorinating agent to obtain the compound having the structure: 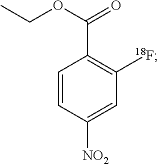
or a process for preparing the compound having the structure: 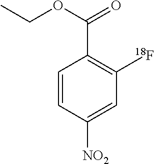
which comprises
(a) reacting 2,4-dinitrobenzonitrile with a [¹⁸F] fluorinating agent to obtain the compound having the structure: 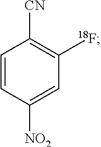
(b) reacting the compound obtained in step (a) with a base to obtain the compound having the structure: 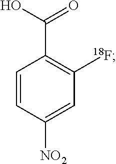
and
(c) esterifying the carboxylic acid group in the compound obtained in step (b) to obtain the compound having the structure:

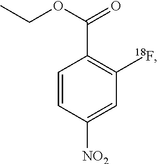

16. (canceled)

17. The process of claim 15, wherein the [¹⁸F] fluorinating agent is potassium [¹⁸F] fluoride or tetra-n-butylammonium [¹⁸F] fluoride, or
a) wherein step (a) further comprises a chelating agent; or wherein step (a) further comprises a base; and/or wherein step (a) is performed at 80-110° C.;
b) wherein in step (b) the hydrolysis is facilitated by aqueous solution of a base; and/or wherein step (b) is performed at a temperature of 80-110° C.; or
c) wherein in step (c) the esterification is facilitated by a base, or wherein in step (c) the esterification agent comprises a toluenesulfonyl group; and/or wherein step (c) is performed at a temperature of 100-120° C.

18. (canceled)

19. The process of part a) of claim 17, wherein the chelating agent is a crown ether, or 4,7,13,16,21,24-Hexaoxa-1,10-diazabicyclo[8.8.8]hexacosane; or the process of part b) of claim 17, wherein step (b) is performed at a temperature of about 105° C.; or the process of part c) of claim 17, wherein step (c) is performed at a temperature of about 110° C.

20-21. (canceled)

22. The process of part a) of claim 17, wherein the base is potassium carbonate; or the process of part b) of claim 17, wherein the base is potassium hydroxide; or the process of part c) of claim 17, wherein the base is potassium hydroxide, sodium carbonate or potassium carbonate; or wherein the toluenesulfonyl group is ethyl tosylate; or wherein the esterification agent comprises a methylsulfonyl group.

23-35. (canceled)

36. The process of claim 22, wherein the methylsulfonyl group is ethyl mesylate.

37. A composition comprising the compound having the structure: 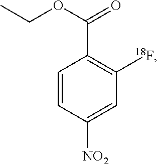
ethyl 2-[¹⁹F]F-4-nitrobenzoate, and at least one acceptable carrier,
wherein the compound is prepared by the process of claim 1.

38. A method of detecting the presence of or location of bacteria cells in a subject which comprises determining if an amount of the compound or determining where an amount of the compound having the structure: 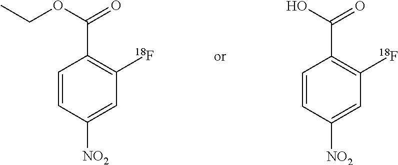
is present in the subject at a period of time after administration of the compound or salt thereof to the subject, thereby detecting the presence of or location of the bacteria cells based on the amount of the compound determined to be present in the subject or detecting the location of the bacteria cells based on the location of the compound determined to be present in the subject.

39. The method of claim 38, further comprising quantifying the amount of the compound in the subject and comparing the quantity to a predetermined control; or further comprising of the compound in the subject, or further comprising subjecting the subject to antibiotic treatment when the presence of or location of bacteria cells is detected.

40. (canceled)

41. The method of claim 38, wherein the presence of the compound is determined by a Positron Emission Tomography (PET) device.

42. The method of claim 38, wherein the bacteria cells express dihydropteroate synthase (DHPS).

43. The method of claim 38, wherein the subject is afflicted with a Gram-negative bacterial infection other than Enterococcus faecalis, or wherein the subject is afflicted with a Gram-positive bacterial infection.

44. (canceled)

45. The method of claim 38, wherein the subject is afflicted with a Mycobacterium tuberculosis bacterial infection, or wherein the subject is afflicted with a Methicillin-sensitive Staphylococcus aureus bacterial infection.

46-48. (canceled)

49. The method of claim 39, wherein the antibiotic is a penicillin, a cephalosporin, a macrolide, a fluoroquinolone, a tetracycline, a carbapenem or an aminoglycoside antibiotic.

50. The method of claim 38, wherein the subject is afflicted with osteomyelitis, or endocarditis, or a prosthetic joint infection, or diabetes.

51-53. (canceled)

54. The method of claim 38, wherein the subject is a mammal.
